(19) United States
(12) Patent Application Publication
(10) Pub. No.: US 2022/0106424 A1
(43) Pub. Date: Apr. 7, 2022

(54) HYDROGEL COMPOSITION FOR A SEMI-RIGID ACOUSTIC COUPLING MEDIUM IN ULTRASOUND IMAGING

(71) Applicant: Decision Sciences Medical Company, LLC, Poway, CA (US)

(72) Inventors: Zachary Staebler, Poway, CA (US); Allan Wegner, Poway, CA (US)

(21) Appl. No.: 17/430,007

(22) PCT Filed: Feb. 13, 2020
(86) PCT No.: PCT/US20/18123

§ 371 Date: Aug. 11, 2021

Related U.S. Application Data
(60) Provisional application No. 62/805,306, filed on Feb. 13, 2019.

(51) Int. Cl. C08F 251/00; C08F 220/56; C08L 5/04; A61B 8/00; C08F 2/10; C08K 3/30; C08K 5/17; C08K 5/20; C08F 2/44; C08F 222/38

(52) U.S. Cl. CPC C08F 251/00; C08F 220/56; C08L 5/04; A61B 8/4281; C08F 2/10; A61B 2562/14; C08K 5/17; C08K 5/20; C08F 2/44; C08F 222/385; C08K 2003/3054; C08K 3/30

(57) ABSTRACT

Disclosed are compositions and articles for a semi-rigid hydrogel material that provides an acoustic coupling medium for ultrasound diagnostic and treatment techniques. In one aspect, a hydrogel material for an acoustic coupling medium includes a sodium alginate block copolymer, a dimethylacrylamide monomer, and water. In some implementations, the sodium alginate block copolymer is present in an amount of about 0.5 wt % to about 25 wt %, the dimethylacrylamide monomer is present in an amount of about 1 wt % to about 40 wt %, and the water is present in an amount of at least about 50 wt % of the total weight of the hydrogel composition.

### CROSS-REFERENCE TO RELATED APPLICATIONS

This patent document claims priorities to and benefits of U.S. Provisional Patent Application No. 62/805,306 entitled “HYDROGEL COMPOSITION FOR A SEMI-RIGID ACOUSTIC COUPLING MEDIUM IN ULTRASOUND IMAGING” filed on Feb. 13, 2019. The entire content of the aforementioned patent application is incorporated by reference as part of the disclosure of this patent document.

### TECHNICAL FIELD

This patent document relates to compositions, articles and methods for an acoustic coupling medium useful for ultrasound imaging.

### BACKGROUND

Acoustic imaging is an imaging modality that employs the properties of sound waves traveling through a medium to render a visual image. High frequency acoustic imaging has been used as an imaging modality for decades in a variety of biomedical fields to view internal structures and functions of animals and humans. High frequency acoustic waves used in biomedical imaging may operate in different frequencies, e.g., between 1 and 20 MHz, or even higher frequencies, and are often termed ultrasound waves. Some factors, including inadequate spatial resolution and tissue differentiation, can lead to less than desirable image quality using conventional techniques of ultrasound imaging, which can limit its use for many clinical indications or applications.

### SUMMARY

Disclosed are compositions and articles for a semi-rigid hydrogel material that provides an acoustic coupling medium for ultrasound diagnostic and treatment techniques. In some aspect, the semi-rigid hydrogel material includes sodium alginate block copolymer and dimethylacrylamide monomer.

In some aspects in accordance with the disclosed technology, a semi-rigid acoustic coupling medium includes a hydrogel material, the hydrogel material comprising: a sodium alginate block copolymer (P(SA)), a dimethylacrylamide monomer (DMAm), and water.

In some aspects in accordance with the disclosed technology, a hydrogel composition includes sodium alginate block copolymer (P(SA)), dimethylacrylamide monomer (DMAm), and water, wherein the P(SA) is present in an amount of about 0.5 wt % to about 25.00 wt %, the DMAm is present in an amount of about 1.00 wt % to about 40.00 wt %, and the water is present in an amount of at least about 50.00 wt % of the total weight of the hydrogel composition.

In some aspects in accordance with the disclosed technology, a hydrogel composition includes sodium alginate block copolymer (P(SA)), dimethylacrylamide monomer (DMAm), and water, wherein the P(SA) is present in an amount of about 0.5 wt % to about 5.5 wt %, the DMAm is present in an amount of about 3.3 wt % to about 14.8 wt %, and the water is present in an amount of at least about 75.6 wt % of the total weight of the hydrogel composition.

In some aspects in accordance with the disclosed technology, a hydrogel composition includes dimethylacrylamide monomer (DMAm), wherein the DMAm is present in an amount of about 1.00 wt % to about 40.00 wt %.

In some aspects in accordance with the disclosed technology, a hydrogel composition includes sodium alginate block copolymer (P(SA)), wherein the P(SA) is present in an amount of about 0.5 wt % to about 25 wt %.

The subject matter described in this patent document can be implemented in specific ways that provide one or more of the following features.

### BRIEF DESCRIPTION OF THE DRAWINGS

FIGS. 1A and 1B show diagrams illustrating a conventional acoustic couplant which exhibits a lack of conformability to a patient's skin.

FIG. 2 shows diagrams illustrating conventional acoustic couplants, which can be comprised of polymers that create rigid gaps between the interface of the coupling medium and the patient's skin.

FIGS. 3A-3D depict the chemical structures of exemplary components of a hydrogel interface pad (HIP) according to an example embodiment of the present disclosure.

FIGS. 4A-4C depict the chemical structures of the polymeric chains of a HIP according to an example embodiment of the present disclosure.

FIG. 4D depicts an exemplary polymeric network, highlighting the ion-ion junctions between the polymeric chains of the HIP according to an example embodiment of the present disclosure.

FIG. 5A shows a schematic depicting an exemplary HIP of the present disclosure undergoing crack propagation testing at stress regions #1, #2, and #3.

FIGS. 5B-5D show more detailed schematics of the exemplary HIP at stress regions #1, #2, and #3 of FIG. 5A, respectively.

FIGS. 6A and 6B show a diagram depicting a super aggregate hydrogel.

FIGS. 7A and 7B show a diagram depicting a weak, brittle hydrogel.

FIG. 8 shows a schematic illustrating acoustic transmission through an example HIP into a heterogenous substrate containing homogenous bodies.

FIG. 9 shows images of example acoustic couplants including example HIPs in accordance with the present technology used in example implementations for evaluating acoustic and mechanical properties of the couplants.

FIGS. 10A-10F show images of example HIPs under mechanical stress.

FIG. 11 shows an image of an example ionically, crosslinked HIP.

FIG. 12 shows an illustrative diagram depicting an example embodiment of a method for synthesizing a hydrogel composition in accordance with the present technology that can be used to produce a HIP.

FIG. 13 depicts a hydrogel network comprising 95% water and the corresponding electron microscopy images.

### DETAILED DESCRIPTION

Acoustic imaging can be performed by emitting an acoustic waveform (e.g., pulse) within a physical elastic medium, such as a biological medium, including tissue. The acoustic waveform is transmitted from a transducer element (e.g., of an array of transducer elements) toward a target volume of interest (VOI). Propagation of the acoustic waveform in the medium toward the target volume can encounter structures that cause the acoustic waveform to become partly reflected from a boundary between two mediums (e.g., differing biological tissue structures) and partially transmitted. The reflection of the transmitted acoustic waveform can depend on the acoustic impedance difference between the two mediums (e.g., at the interface between two different biological tissue types). For example, some of the acoustic energy of the transmitted acoustic waveform can be scattered back to the transducer at the interface to be received, and processed to extract information, while the remainder may travel on and to the next medium. In some instances, scattering of the reflection may occur as the result of two or more impedances contained in the reflective medium acting as a scattering center. Additionally, for example, the acoustic energy can be refracted, diffracted, delayed, and/or attenuated based on the properties of the medium and/or the nature of the acoustic wave.

Acoustic wave speed and acoustic impedance differences can exist at the interface between the transducer and the medium to receive the acoustic waveform, e.g., referred to as the receiving medium, for propagation of the acoustic waveform toward the target volume, which can disrupt the transmission of the acoustic signal for imaging, range-Doppler measurement, tissue characterization (e.g., Acoustic Radiation Force Impulse—ARFI), or therapeutic applications. Acoustic impedance differences caused due to differing material properties (e.g., material density) of the two mediums and the acoustic wave velocity, such that a substantial amount of the emitted acoustic energy will be reflected at the interface rather than transferred in full across the interface. In typical acoustic (e.g., ultrasound) imaging or therapy applications, for example, a transmission gel is applied to the receiving medium (i.e., the skin of a subject) at the interface where the transducers will make contact to improve the transfer of the acoustic waveform(s) from the transducer to the body and the reception of the returned acoustic waveform(s) from the body back to the transducer. In such applications without the ultrasound gel, the interface may include air as a component of the medium between the receiving medium (e.g., living skin tissue) and the transducer, and an acoustic impedance mismatch in the transducer-to-air and the air-to-body discontinuity causes the scattering (e.g., reflection) of the emitted acoustic energy.

Despite relatively good success in reducing acoustic impedance difference at the interface, when dispensed on the VOI, acoustic transmission gels may contain tiny packets of air that can disrupt the transmission of acoustic signals. Additionally, many patients complain of discomforts with the use of gels dispensed on their skin, e.g., such as temperature, stickiness, or other. More concerning, however, acoustic transmission gels can become contaminated during production or storage, which has led to infections within some patients. For subjects with hair on their skin at the location where the transducer is to be placed, these subjects typically must shave or otherwise remove the external hair which exasperates the trapping of air between the skin and gel.

For non-normal angles of incidence of the acoustic wave relative to the interface, the differences in the acoustic wave speed can result in refraction of the acoustic sound wave. Acoustic wave speed differences at the interface cause the propagation path of longitudinal acoustic waves to refract or change direction according to Snell's Law as a function of the angle of incidence and the acoustic wave speeds either side of the interface. Accumulations of infinitesimal amounts of refraction as the wave propagates in a heterogeneous material results in bending or curvature in the path of the acoustic wave.

As conventional ultrasound (US) imaging assumes that acoustic waves travel in straight lines, refraction along the acoustic path causes degradation and distortion in the resulting image due the ambiguity it creates for the arrival time and location of an acoustic waveform in space for both transmission and reception. A material that matches the acoustic wave speed at the interface significantly reduces the effects of refraction, resulting in a clearer and less ambiguous image. Additionally, a semi-rigid material that has a homogeneous acoustic wave speed throughout will minimize the potential for curvature of acoustic wave paths inside the material.

Ultrasound imaging gained interest in the medical imaging community for portability, multiple anatomic target modalities, safety, and relatively low cost when compared to X-ray, computerized tomography (CT), and magnetic resonance imaging (MRI) techniques. Some modalities focus entirely on cardiology and can create 4-D images of beating ventricles. Other modalities are dedicated calculators that compute fluid flow through tiny corpuscular capillaries in the liver and spleen whereas other modalities simply use the US as a general-purpose machine. Regardless how narrow or broad the application, all US machines suffer from the same limitations engendered from traditional ultrasound design, i.e., loss of image quality at depth and low near field resolution. While the image depth depends mostly on array design and transducer frequency, the obfuscated near field is the result of large impedance mismatch differences between the transducer interface and patient interface and the focal point of the transducer.

Near field convolution is an annoyance encountered in many US diagnostic techniques, especially for synovial joints which are bundles of tendon, fluid, bone, and muscle tightly bound together under a thin, sinewy veil of skin and tissue. This is a ubiquitous problem, and many clinicians have resorted to filling a nitrile rubber glove with tap water to act as a portable, quasi water bath that doubled as a standoff, e.g., any acoustic coupling material providing distance between the transducer interface and patient interface. Simple, cost effective, and fast to implement, this artifice was good enough solution for generating quick non-visceral US images with linear arrays.

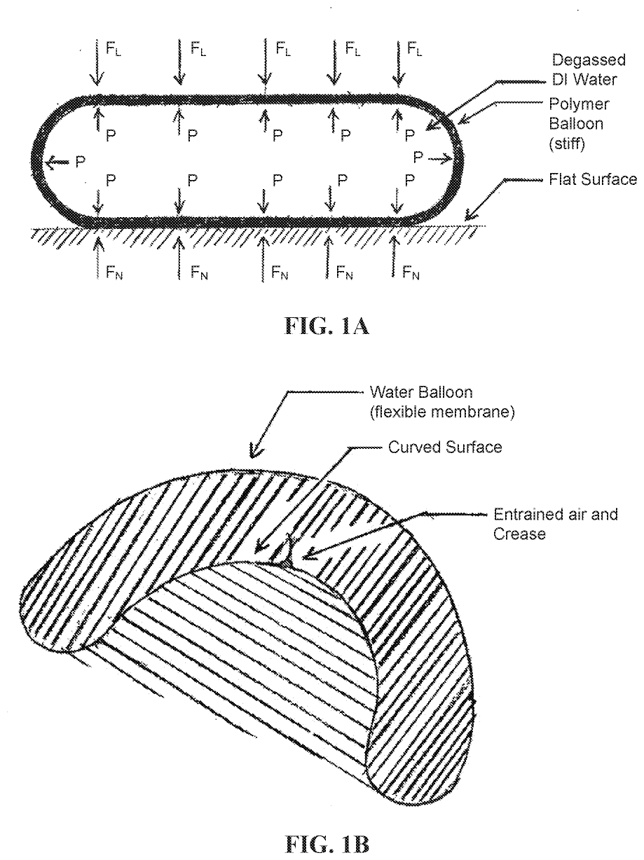

FIGS. 1A and 1B show diagrams illustrating an acoustic couplant which exhibits a lack of conformability to a patient's skin, such as a conventional water balloon couplant. As shown FIG. 1A, the water balloon couplant, in this example, includes a polymer balloon outer membrane that encompasses degassed water (e.g., degassed deionized (DI) water) within the polymer outer membrane. The degassed water entrapped within the outer membrane provides a pressure on the inner surface of the outer membrane, such that the shape of the water balloon couplant is defined by the external forces exerted upon the water balloon—in this example, the external forces include a normal force (F_{N}) exerted by a flat surface in contact with the water balloon couplant and outer force (F_{L}) from the outer environment. The outer membrane of the water balloon couplant is typically flexible, and can be bent to attempt to fit around curved surfaces, as shown by the diagram in FIG. 1B. However, such bending typically creates entrained air and creases at inflexion points along the outer membrane and within the fluidic interior of the water balloon couplant.

Furthermore, for non-linear arrays and non-planar surfaces, technical issues become too challenging for simple water balloons to surmount. Take for an instance a semicircular array for Acoustic Coherent Tomography (ACT) which has several array elements that need to couple to a swath of variegated patient interface geometries during a multi-VOI examination. The first challenge with water balloons is contorting the tubular geometry to couple to the transducer interface without creasing on the patient interface, as shown in FIG. 1. Creases will trap air which look like comets in US images with bright spots that shadow out anatomic features and generate artifacts. Even if a few mil-thick (e.g., 0.001 inch-thick) polymer membrane was designed to fit in the array without creasing, the water balloon still lacks conformability needed to scan multiple anatomic targets in a single examination since water is a semi-incompressible fluid (k=46.4×10⁻⁶ atm⁻¹) and conservation of volume principles apply.

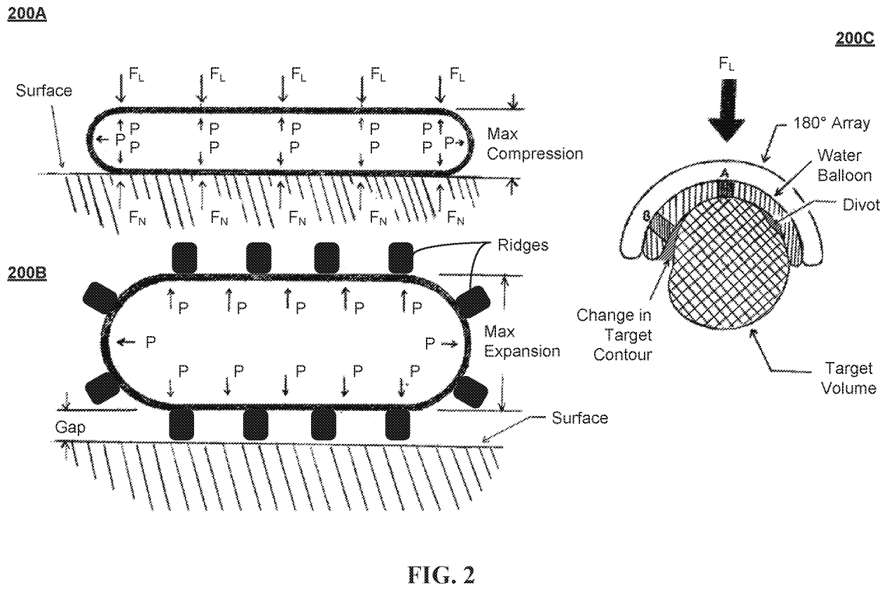

For polymers with thick walls, high young modulus, and low strain before failure the load on the transducer side of the water balloon is directly transmitted to the patient interface without dispersing the load over a larger surface area and without conforming to the non-symmetric patient geometry. Low elastic modulus, high strain before failure, and thin walled polymers might deform more, but are not conformable enough to bridge large gaps between the rigid, symmetrical transducer interface and the asymmetric, deformable patient interface, and are more prone to bursting and rolling during examinations, as illustrated in FIG. 2.

FIG. 2 shows diagrams illustrating a conventional acoustic couplant, such as a water balloon couplant, that is comprised of polymers that create rigid gaps between the interface of the coupling medium and the patient's skin. Diagram 200A shows the example water balloon acoustic couplant in contact with a surface, illustrating maximum compression on the water balloon couplant between applied external forces from a surface in contact with the couplant (normal force F_{N}) and forces (F_{L}) from the surrounding environment. Diagram 200B shows the example water balloon acoustic couplant that includes ridges in the outer polymer membrane that creates a gap between a contact surface, also showing the example water balloon acoustic couplant under maximum expansion. Diagram 200C shows an example water balloon couplant in contact with a target volume (e.g., patient's skin of a body part), illustrating how the water balloon couplant may have gaps between the couplant and target volume due to divots and/or changes in the contour of the target.

A more conformable and durable standoff was needed, so thin, semisolid, hydrogel pucks or sheets (e.g., ˜1.0-1.5 cm) have been developed to accommodate traditional US imaging in the near field. These hydrogel puck or sheet standoffs aim at minimizing the impedance mismatch between the rigid, symmetrical transducer interface and the asymmetrical, conformable patient interface for linear arrays. More conformable than water balloons, thin hydrogel sheets can fill in divots and escarpments along planar surfaces and form to eclectic curved topography. Additionally, depending on the hydrogel chemistry and morphology, hydrogels can either be sticky for long, static US diagnostic scans or generate a lubricating layer via syneresis when conducting short, dynamic scans under pressure.

Yet, despite greater conformability than water balloons, hydrogels on the current market have a large bulk modulus which increases hydrogel rigidity as the thickness increases. Coupled with low fracture toughness and paraben preservatives, the stiffness and brittleness, the ease of crack propagation, and the ambiguity of health safety render hydrogel standoffs useless in applications where a thick (>2 cm), tough, and conformable semi-rigid standoff is needed for non-linear arrays like the aforementioned ACT semicircular array.

Disclosed are compositions and articles for a semi-rigid hydrogel material that provides an acoustic coupling medium for ultrasound techniques. The use of a hydrogel coupling medium in accordance with the embodiments disclosed herein offers advantages over conventional coupling mediums such as water baths and standoffs like water bags and puck or sheet hydrogels, e.g., including, but not limited to, offering superior acoustic and mechanical properties, as well as enabling low manufacturing costs, rapid rate of production, and minimized surplus.

In some aspects, the disclosed semi-rigid hydrogels include an engineered polymer network having the ability to form elaborate geometries and entrap water to a high percentage (e.g., 85% or greater) that provides acoustic impedance matching between ultrasound transducer elements and the target biological volume. The disclosed hydrogels provide additional advantages in their manner of manufacture, distribution and application based on their low-cost of fabrication, simultaneous step of sterilization and curing, stable storage, and biocompatibility.

Also disclosed are compositions and articles for a rigid hydrogel material that provides an acoustic coupling medium useful for some ultrasound techniques and applications, e.g., particularly as an acoustic couplant between a flat transducer array and a flat surface of a target volume for imaging.

Example Hydrogel Compositions

In some embodiments in accordance with the present technology, a composition of a hydrogel includes a monomer, block copolymer, a dispersive phase, a covalent crosslinker, cationic crosslinking agent, catalyst, and/or a free radical initiator.

A function of the monomer is to serve as the primary, structural network for the hydrogel. In some embodiments, the monomer is an acrylamide. Non-limiting examples of acrylamide monomers include dimethylacrylamide (DMA), diethylacrylamide (DEAA), phenyl acrylamide, tert-butylacrylamide, octadecylacrylamide, isopropylacrylamide, or diphenylmethylacrylamide.

In some embodiments, the monomer is present in an amount between about 1 wt % to about 40 wt %, about 10 wt % to about 40 wt %, about 20 wt % to about 40 wt %, about 30 wt % to about 40 wt %, about 1 wt % to about 10 wt %, about 1 wt % to about 5 wt %, about 1 wt % to about 15 wt %, about 1 wt % to about 20 wt %, about 1 wt % to about 30 wt %, about 10 wt % to about 20 wt %, about 10 wt % to about 30 wt %, about 10 wt % to about 15 wt %, about 15 wt % to about 20 wt %, about 15 wt % to about 30 wt %, about 15 wt % to about 40 wt %, about 20 wt % to about 30 wt %, or about 20 wt % to about 40 wt % of the total weight of the hydrogel composition. In some embodiments, the monomer is DMA as in present in an amount of about 1 wt % to about 40 wt % of the total weight of the hydrogel composition.

In some embodiments, the monomer is present in an amount between about 1 to about 10 wt %, about 1 to about 15 wt %, about 2 wt % to about 10 wt %, about 2 wt % to about 15 wt %, about 3 wt % to about 10 wt %, about 3 wt % to about 15 wt %, about 4 wt % to about 10 wt %, about 4 wt % to about 15 wt %, about 5 wt % to about 10 wt %, about 5 wt % to about 15 wt %, about 6 wt % to about 10 wt %, about 6 wt % to about 15 wt %, about 7 wt % to about 10 wt %, about 7 wt % to about 15 wt %, about 8 wt % to about 10 wt %, about 8 wt % to about 15 wt %, to about 9 wt % to about 10 wt %, or about 9 wt % to about 15 wt % of the total weight of the hydrogel composition. In some embodiments, the monomer is DMA and is present in an amount of about 3.3 wt % to about 14.83 wt % of the total weight of the hydrogel composition.

A function of the block copolymer is to provide a secondary, grated sacrificial network for the hydrogel. In some embodiments, the block copolymer is an alginate. Non-limiting examples of alginates include sodium alginate (SA), potassium alginate, calcium alginate, ammonium alginate, low acetylated gellan gum, high acetylated gellan gum, modified starches, agar, k-Carrageenan, I-Carrageenan, low methoxy pectin, high methoxy pectin, methyl cellulose, hydroxypropyl methyl cellulose, cellulose/gelatin, or propylene glycol alginate.

In some embodiments, the block copolymer is present in an amount between about 0.5 wt % to about 25 wt %, about 0.5 wt % to about 20 wt %, about 0.5 wt % to about 15 wt %, about 0.5 wt % to about 10 wt %, about 0.5 wt % to about 5 wt %, about 0.5 wt % to about 1 wt %, about 1 wt % to about 25 wt %, about 1 wt % to about 20 wt %, about 5 wt % to about 15 wt %, about 5 wt % to about 10 wt %, about 10 wt % to about 25 wt %, about 10 wt % to about 20 wt %, about 10 wt % to about 15 wt %, about 15 wt % to about 25 wt %, or about 15 wt % to about 20 wt % of the total weight of the hydrogel composition. In some embodiments, the block copolymer is SA and is present in an amount about 0.5 wt % to about 25 wt % of the total weight of the hydrogel composition.

In some embodiments, the block copolymer is present in an amount between about 0.1 to about 10 wt %, about 0.1 to about 8 wt %, about 0.1 wt % to about 6 wt %, about 0.1 wt % to about 4 wt %, about 0.1 wt % to about 2 wt %, about 0.2 wt % to about 10 wt %, about 0.2 wt % to about 8 wt %, about 0.2 wt % to about 6 wt %, about 0.2 wt % to about 4 wt %, about 0.2 wt % to about 2 wt %, about 0.3 wt % to about 10 wt %, about 0.3 wt % to about 8 wt %, about 0.3 wt % to about 6 wt %, about 0.3 wt % to about 4 wt %, about 0.3 wt % to about 2 wt %, about 0.4 wt % to about 10 wt %, to about 0.4 wt % to about 8 wt %, about 0.4 wt % to about 6 wt %, about 0.4 wt % to about 4 wt %, about 0.4 wt % to about 2 wt %, about 0.5 wt % to about 10 wt %, to about 0.5 wt % to about 8 wt %, about 0.5 wt % to about 6 wt %, about 0.5 wt % to about 4 wt %, or about 0.5 wt % to about 2 wt % of the total weight of the hydrogel composition. In some embodiments, the block copolymer is SA and is present in an amount about 0.51 wt % to about 5.53 wt % of the total weight of the hydrogel composition.

In some embodiments, the dispersive phase is water (e.g., deionized water). In some embodiments, the dispersive phase is present in an amount of at least about 40 wt %, at least about 50 wt %, at least about 60 wt %, 70 wt %, at least about 75 wt %, at least about 80 wt %, at least about 85 wt %, at least about 90 wt %, or at least about 95 wt % of the total weight of the hydrogel composition. In some embodiments, the dispersive phase is water and is present in an amount of at least about 50 wt % of the total weight of the hydrogel composition. In some embodiments, the dispersive phase is water and is present in an amount of about 75.65 wt % to about 95.98 wt % of the total weight of the hydrogel composition.

In some embodiments the covalent crosslinker is an acrylamide. Non-limiting examples of acrylamide covalent crosslinkers include N′,N′-methylene bisacrylamide (MBA), bisacrylamide, ethylene bisacrylamide, piperazine diacrylamide, or ethylene glycol bisacrylamide.

In some embodiments, the covalent crosslinker is present in an amount between about 0.04 wt % to about 10 wt %, about 0.04 wt % to about 9 wt %, about 0.04 wt % to about 8 wt %, about 0.04 wt % to about 7 wt %, about 0.04 wt % to about 6 wt %, about 0.04 wt % to about 5 wt %, about 0.04 wt % to about 4 wt %, about 0.04 wt % to about 3 wt %, about 0.04 wt % to about 1 wt %, about 1 wt % to about 10 wt %, about 2 wt % to about 10 wt %, about 3 wt % to about 10 wt %, about 4 wt %, to about 10 wt %, about 5 wt % to about 10 wt %, about 6 wt % to about 10 wt %, about 7 wt % to about 10 wt %, about 8 wt % to about 10 wt %, or about 9 wt % to about 10 wt % of the total weight of the hydrogel composition. In some embodiments, the covalent crosslinker is MBA and is present in an amount between about 0.04 wt % to about 10 wt % of the total weight of the hydrogel composition.

In some embodiments, the covalent crosslinker is present in an amount between about 0.01 to about 5 wt %, about 0.01 to about 4 wt %, about 0.1 wt % to about 3 wt %, about 0.02 wt % to about 5 wt %, about 0.02 wt % to about 4 wt %, about 0.02 wt % to about 3 wt %, about 0.02 wt % to about 2 wt %, about 0.03 wt % to about 5 wt %, about 0.03 wt % to about 4 wt %, about 0.03 wt % to about 3 wt %, about 0.3 wt % to about 2 wt %, about 0.04 wt % to about 5 wt %, about 0.04 wt % to about 4 wt %, about 0.04 wt % to about 3 wt %, about 0.04 wt % to about 2 wt %, about 0.05 wt % to about 5 wt %, to about 0.05 wt % to about 4 wt %, about 0.05 wt % to about 3 wt %, about 0.05 wt % to about 2 wt %, about 0.05 wt % to about 5 wt %, about 0.05 wt % to about 4 wt %, to about 0.05 wt % to about 3 wt %, or about 0.05 wt % to about 2 wt % of the total weight of the hydrogel composition. In some embodiments, the covalent crosslinker is MBA and is present in an amount between about 0.041 wt % to about 3.43 wt % of the total weight of the hydrogel composition.

In some embodiments, the cationic crosslinking agent is a monovalent, divalent, trivalent metal. For example, a cationic crosslinking agent can be a transition metal, an alkali metal, or an alkaline earth metal where the metal is the 1⁺, 2⁺, or 3⁺ oxidation state. In some embodiments, the cationic crosslinking agent is lithium, sodium, potassium, magnesium, calcium, zinc, zirconium, iron, cobalt, nickel, titanium, or copper. In some embodiments, the cationic crosslinking agent is in the form of any monovalent divalent, or trivalent salt. For example, in some embodiments the cationic crosslinking agent is any sulfate, phosphate, chloride, bromide, triflate, amine, or carboxylate salt. In some embodiments, the cationic crosslinking agent is calcium sulfate (CA), calcium phosphate, calcium chloride, calcium bromide, or calcium triflate.

In some embodiments, the cationic crosslinking agent is present in an amount between about 0.1 wt % to about 0.5 wt %, about 0.1 wt % to about 0.6 wt %, about 0.1 wt % to about 0.7 wt %, about 0.01 wt % to about 0.8 wt %, about 0.01 wt % to about 0.9 wt %, or about 0.01 wt % to about 1 wt % of the total weight of the hydrogel composition. In some embodiments, the cationic crosslinking agent is CA and is present in an amount between about 0.14 wt % to about 0.23 wt % of the total weight of the hydrogel composition.

A function of the catalyst is to promote and/or increase the rate of the chemical reaction that forms the hydrogel composition. In some embodiments, the catalyst is an amine. Non-limiting examples of amine catalyst include aliphatic amines, N′,N′,N,N-tetramethylethylenediamine (TMED), benzyldimethylamine, methylamine, or triethyl amine.

In some embodiments, the catalyst is present in an amount between about 0.004 wt % to about 1.00 wt %, about 0.004 wt % to about 0.9 wt %, about 0.004 wt % to about 0.8 wt %, about 0.004 wt % to about 0.7 wt %, about 0.004 wt % to about 0.6 wt %, about 0.004 wt % to about 0.5 wt %, about 0.004 wt % to about 0.4 wt %, about 0.004 wt % to about 0.3 wt %, about 0.004 wt % to about 0.2 wt %, about 0.004 wt % to about 1 wt %, 0.01 wt % to about 1 wt %, about 0.1 wt % to about 1 wt %, about 0.2 wt % to about 1 wt %, about 0.3 wt % to about 1 wt %, about 0.4 wt % to about 1 wt %, about 0.5 wt % to about 1 wt %, about 0.6 wt % to about 1 wt %, about 0.7 wt % to about 1 wt %, about 0.8 wt % to about 1 wt %, or about 0.9 wt % to about 1 wt % of the total weight of the hydrogel composition. In some embodiments, the catalyst is TMED and is present in an amount between about 0.004 wt % to about 1 wt % of the total weight of the hydrogel composition.

In some embodiments, the catalyst is present in an amount between about 0.001 wt % to about 0.05 wt %, about 0.001 wt % to about 0.06 wt %, about 0.001 wt % to about 0.07 wt %, about 0.001 wt % to about 0.08 wt %, about 0.001 wt % to about 0.09 wt %, or about 0.001 wt % to about 0.1 wt % of the total weight of the hydrogel composition. In some embodiments, the catalyst is TMED and is present in an amount between about 0.004 wt % to about 0.08 wt % of the total weight of the hydrogel composition.

A function of the free radical initiator is to generate free radicals that initiate the formation of the polymeric network of the hydrogel composition. Non-limiting examples of free radical initiators includes ammonium persulfate (APS), ribofalvin-5′-phosphate, ribofalvin-5′-phosphate sodium, peroixdes such as dialkyl peroxides, hydroperoxides, diacyl periods, or azo-compounds (i.e., —N═N— moieties). In some embodiments, the initiator is a photoinitiator. Non-limiting examples of photo initiators include ethyl (2,4,5-trimethylbenzoyl) phenyl phosphinate (TPO-L), bis-acylphosphine oxide (BAPO), 2-hydroxy-2-methyl propiophenone, methylbenzoyl formate, isoamyl 4-(dimethylamino) benzoate, 2-ethyl hexyl-4-(dimethylamino) benzoate, or diphenyl(2,4,6-trimethylbenzoyl) phosphine oxide (TPO). Additional, non-limiting examples of suitable photo-initiators include 1-hydroxycyclohexyl phenyl ketone (Irgacure 184), 2,2-dimethoxy-2-phenylacetophenone (Irgacure 651), and 2-methyl-1-[4-(methylthio) phenyl]-2-(4-morpholinyl)-1-propanone (Irgacure 907), hydroxyacetophenone, phosphineoxide, benzophenone, and lithium phenyl-2,4,6-trimethylbenzoylphosphinate (LAP).

In some embodiments, the initiator is present in an amount between about 0.01 wt % to about 1 wt %, about 0.01 wt % to about 0.9 wt %, about 0.01 wt % to about 0.8 wt %, about 0.01 wt % to about 0.7 wt %, about 0.01 wt % to about 0.6 wt %, about 0.01 wt % to about 0.5 wt %, about 0.01 wt % to about 0.4 wt %, about 0.01 wt % to about 0.3 wt %, about 0.01 wt % to about 0.2 wt %, about 0.01 wt % to about 0.1 wt %, about 0.02 wt % to about 1 wt %, about 0.03 wt % to about 1 wt %, about 0.04 wt % to about 1 wt %, about 0.05 wt % to about 1 wt %, about 0.06 wt % to about 1 wt %, about 0.07 wt % to about 1 wt %, about 0.08 wt % to about 1 wt %, about 0.09 wt % to about 1 wt %, or about 0.1 wt % to about 1 wt % of the total weight of the hydrogel composition. In some embodiments, the initiator is APS and is present in an amount between about 0.01 wt % to about 1 wt % of the total weight of the hydrogel composition.

In some embodiments, the initiator is present in an amount between about 0.01 wt % to about 0.05 wt %, about 0.01 wt % to about 0.06 wt %, about 0.01 wt % to about 0.07 wt %, about 0.01 wt % to about 0.08 wt %, about 0.01 wt % to about 0.09 wt %, about 0.01 wt % to about 0.1 wt %, 0.02 wt % to about 0.15 wt %, about 0.02 wt % to about 0.16 wt %, about 0.02 wt % to about 0.17 wt %, about 0.02 wt % to about 0.18 wt %, about 0.02 wt % to about 0.19 wt %, about 0.01 wt % to about 0.2 wt %, or about 0.02 wt % to about 0.25 wt % of the total weight of the hydrogel composition. In some embodiments, the initiator is APS and is present in an amount between about 0.02 wt % to about 0.24 wt % of the total weight of the hydrogel composition.

In some embodiments, the hydrogel comprises a dimethyl acrylamide monomer (DMAm), a sodium alginate block copolymer (P(SA)), and water. In some embodiments, the hydrogel composition further comprises MBA, TMED, CA, and APS. In some embodiments, the DMAm is present in an amount between about 3.3 wt % to about 14.83 wt % of the total weight of the hydrogel composition; and in some embodiments the DMAm is present in an amount of about 8.3 wt % to about 9.8 wt %. For example, the DMA concentration can be engineered to affect the elasticity and conformability. In some embodiments, the P(SA) is present in an amount between about 0.52 wt % to about 5.53 wt % of the total weight of the hydrogel composition. In some embodiments, the MBA is present in an amount of between about 0.041 wt % to about 3.44 wt % of the total weight of the hydrogel composition. In some embodiments, the TMED is present in amount between about 0.004 wt % to about 0.02 wt % of the total weight of the hydrogel composition. In some embodiments, the CA is present in an amount between about 0.14 wt % to about 0.23 wt % of the total weight of the hydrogel composition. In some embodiments, the APS is present in an amount between about 0.0198 wt % to about 0.235 wt % of the total weight of the hydrogel composition. In some embodiments, the hydrogels further comprise water (e.g., degassed water) in an amount between about 75.65 wt % to about 95.98 wt % of the total weight of the hydrogel composition.

In some embodiments, the hydrogel comprises a dimethyl acrylamide monomer (DMAm), a sodium alginate block copolymer (P(SA)), and water. In some embodiments, MBA, TMED, CA, and APS. In some embodiments, the DMAm is present in an amount between about 3.3 wt % to about 14.83 wt % of the total weight of the hydrogel composition; and in some embodiments the DMAm is present in an amount of about 1 wt % to about 40 wt %. For example, the DMA concentration can be engineered to affect the elasticity and conformability. In some embodiments, the P(SA) is present in an amount between about 0.5 wt % to about 25 wt % of the total weight of the hydrogel composition. In some embodiments, the MBA is present in an amount of between about 0.04 wt % to about 10 wt % of the total weight of the hydrogel composition. In some embodiments, the TMED is present in amount between about 0.004 to about 1 wt % of the total weight of the hydrogel composition. In some embodiments, the CA is present in an amount between about 0.01 wt % to about 1 wt % of the total weight of the hydrogel composition. In some embodiments, the APS is present in an amount between about 0.01 wt % to about 1 wt % of the total weight of the hydrogel composition. In some embodiments, the hydrogels further comprise water (e.g., degassed water) in an amount at least about 50 wt % of the total weight of the hydrogel composition.

In some example implementations, the hydrogel composition can be formed as a hydrogel interface pad that includes more than 91 wt % water at the expense of other constituents (e.g., P(SA) or DMAm) to yield a more fragile hydrogel pad, e.g., including 95 w % water hydrogel interface pad.

Example Embodiments of a Hydrogel Interface Pad (HIP)

In various embodiments, the example hydrogel compositions can be configured in a semi-rigid pad, referred to herein as a hydrogel interface pad (HIP). In some exemplary embodiments of the present disclosure, the HIP is made up of two water soluble polymer networks: a primary (1°) scaffold and a secondary (2°) sacrificial graft.

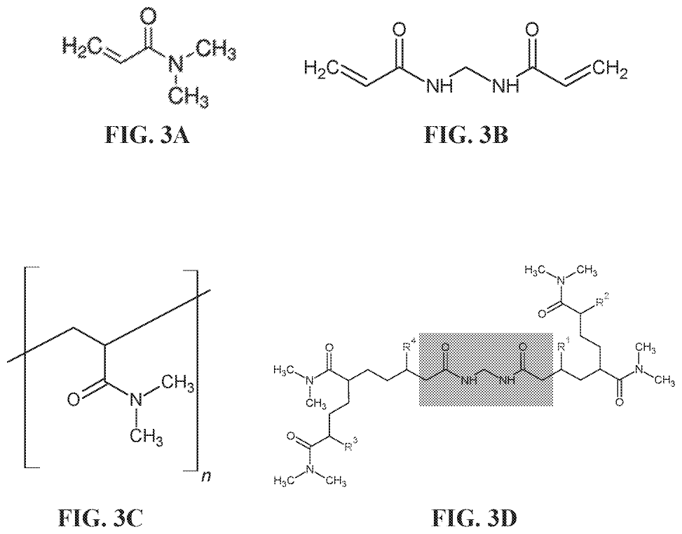

FIGS. 3A-3D depict the chemical structures of components of an example HIP. FIG. 3A is the chemical structure of DMA, and FIG. 3B is the chemical structure of MBA. The 1° scaffold is composed of DMA monomers as shown in FIG. 3C polymerized via free radical vinyl addition and crosslinked by MBA monomers (MBAm) as shown in FIG. 3D. DMA monomer is used instead of DMA terminated polymer to yield an extensively crosslinked polymer with long chain lengths for strength and elasticity. Terminated poly(DMAm) chains crosslinked via vinyl addition reactions yield polymers with shorter chain lengths resulting in brittle, rigid hydrogels. Extensive crosslinking and extent of polymerization in the 1° network increases the elasticity and conformability of the poly(DMAm) hydrogel while increasing the burst pressure. Additional strength is achieved through intramolecular interactions, like H-bonding, and polymer entanglements.

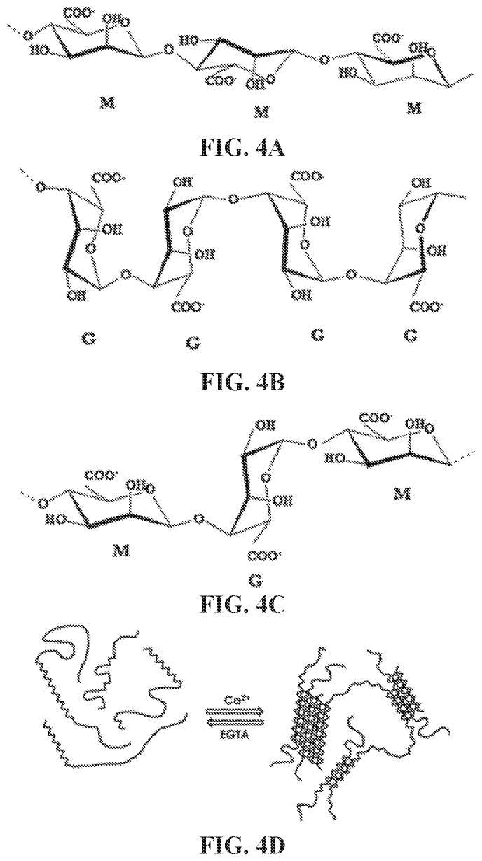

FIGS. 4A-4C depict the chemical structures of the polymeric chains of an example HIP. The 2° networks are composed of terminated chains of P(SA) block copolymer which are further composed linear of β-d-mannuronate (M) (See FIG. 4A) and kinked α-1-guluronate (G) (See FIG. 4B) homogenous block polymers and heterogenous, bent Poly(MG) (See FIG. 4C) block polymers.

FIG. 4D depicts a polymeric network, highlighting the ion-ion junctions between the polymeric chains of the example HIP. The resulting polymer is a series of amorphic regions connected to a series of ordered regions engaged in ion-ion junctions with configurations similar to egg cartons, as shown in FIG. 4D. High molecular weight chains generally have higher viscosities which increase SA hydrogel strength and turbidity from more chain entanglements and ion-ion junctions, while low molecular weight chains have lower viscosities which improves gel clarity at the expense of gel strength due to less ion-ion junctions and entanglements.

The SA hydrogel mechanical properties can also be tuned by adjusting the P(SA) block polymer composition of G-blocks and M-blocks. High G-block concentrations increase the SA hydrogel turbidity, rigidity, and strength by increasing the chain ion-ion junction density, while low G-block concentrations increase SA hydrogel clarity, elasticity, and brittleness by reducing the chain ion-ion junction density.

Under tension, DMAm hydrogels undergo significant elastic deformation before plastic deformation shortly followed by rapid failure. Low fracture toughness causes rapid failure in the plastic region, resultant of precipitated rupture of covalent crosslinks and poly(DMAm) chains around localized stress sites, such as small fractures and crazing, which propagate as large cracks.

Under compression, SA hydrogels will undergo plastic deformation by unzipping the G-blocks around the divalent cations to dissipate stress which induces strain hardening as the polymer structures compress. Once the compressive load is removed the G-blocks will reform, or “re-zip”, around the divalent cations. Like DMAm hydrogels, SA alginate hydrogels have low fracture toughness because ion-ion junctions are readily broken at localized stress regions.

Since poly(DMAm) and P(SA) hydrogels both have low fracture toughness, neither can be used as a sonolucent standoff and acoustic couplant withstanding clinical application. But, combined, the HIP Double Interpenetrating Network (DIN) mechanical properties are greater than the sum of its parts. Grafting P(SA) to poly(DMAm) via covalent bonding dissipates localized stresses across the bulk of the material while preserving the poly(DMAm) backbone.

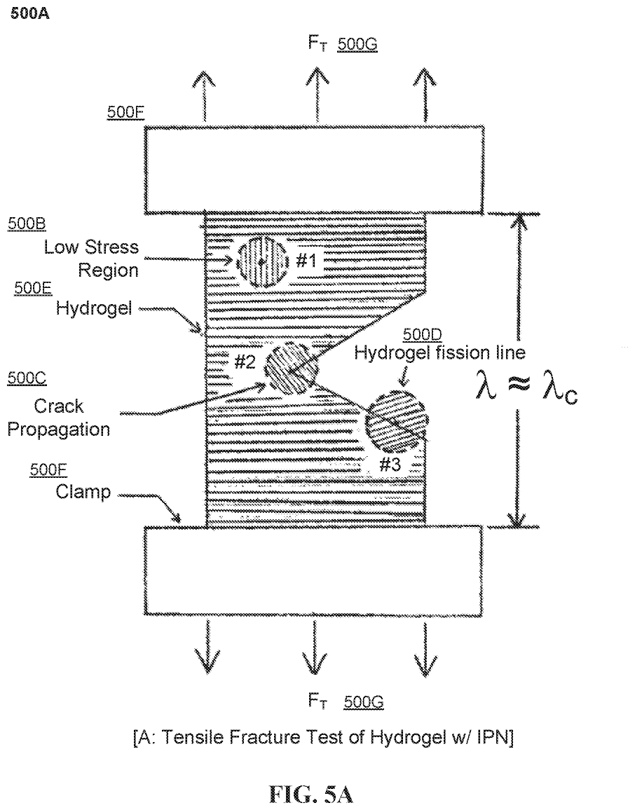

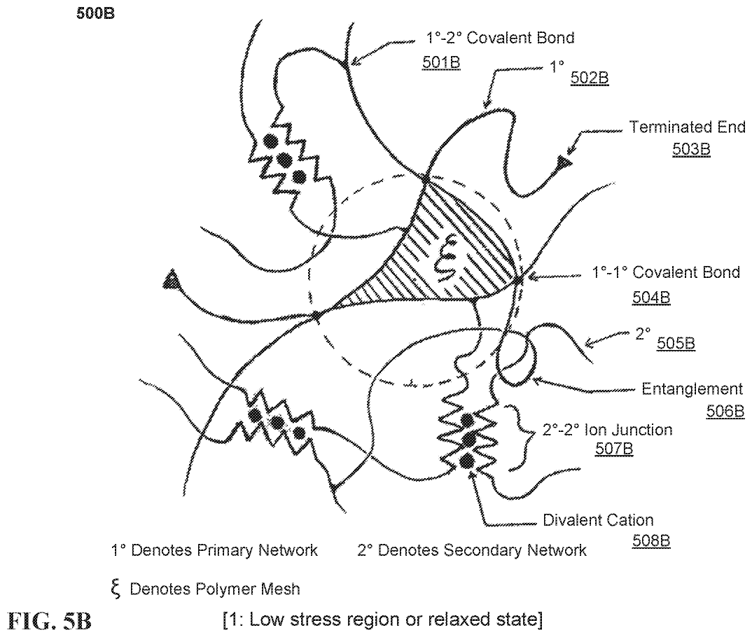

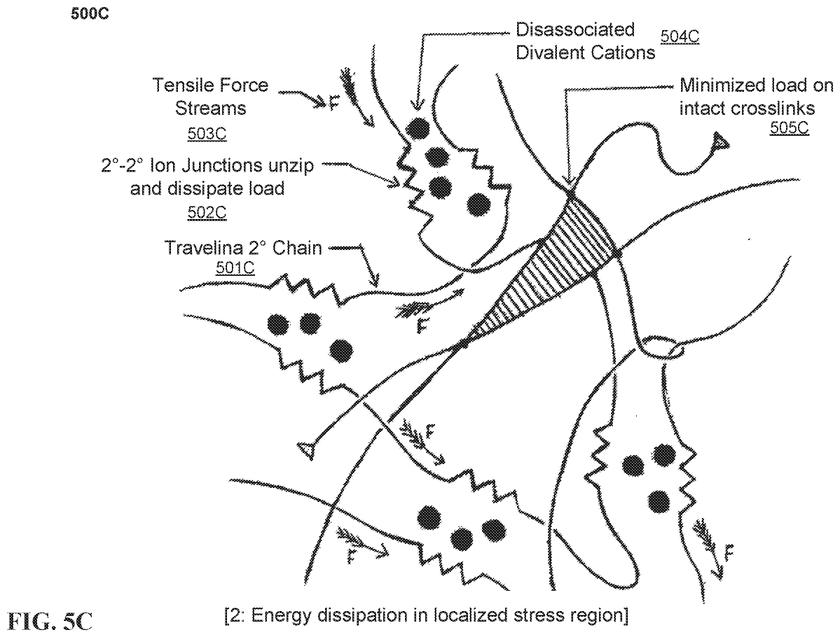

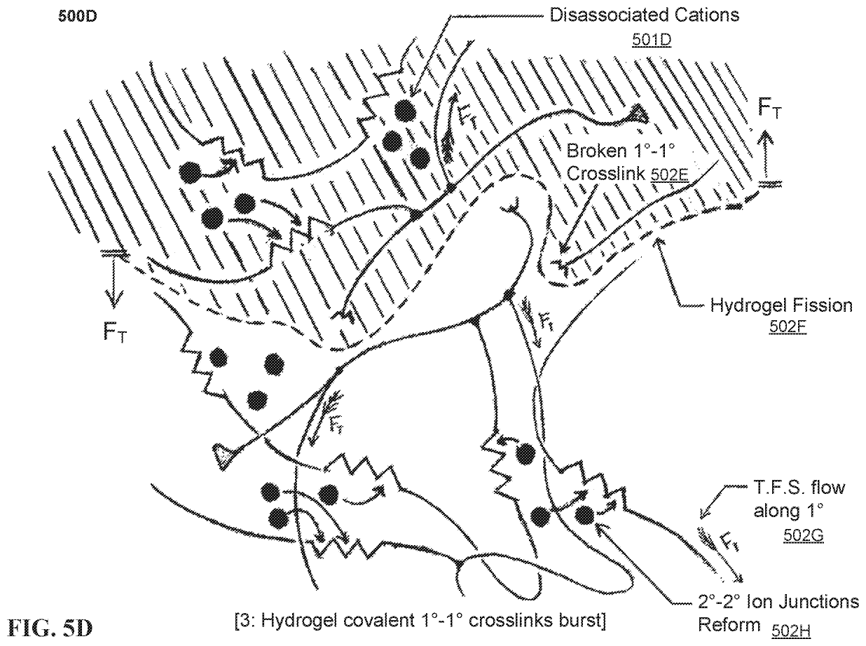

FIGS. 5A-5D show a set of illustrations of the 1° and 2° network morphology and its correlation to localized stress dispersion and fracture toughness is presented for a crack initiated HIP specimen at the crack propagation strain (λ_{c}) under a tensile load. Specifically, FIG. 5A is a schematic of the HIP undergoing crack propagation testing (labeled 500A), where stress occurs at three circle regions referred as region (#1) denoted by 500B, region (#2) denoted by 500C, and region (#3) denoted by 500D. Region 500B refers to a low stress region of the hydrogel 500E, region 500C refers to the cracked propagation region of the hydrogel 500E, and lastly, region 500D refers to the hydrogel fission line of the hydrogel 500E. The stress occurs when the hydrogel 500E is placed between the clamps 500F and a force 500G is applied to the hydrogel 500E, resulting in the stress regions 500B, 500C, and 500D. FIGS. 5B-5D are zoomed in depictions of the stress regions #1, #2, and #3, respectively.

FIG. 5B represents the low stress region #1 (labeled 500B) depicted in FIG. 5A. In the diagram of FIG. 5B, 502B denotes the 1° network with 503B representing the terminal end of the 1° network. 505B denotes the 2° network. 501B is the covalent bond between the 1° network 502B and 2° network 505B. 504B represents covalent bonding between the 1° network 502B. 506B represents an entanglement between the 1° network 502 and 2° network 505B, and 507B represents an ion-ion junction formed between the 2° network with intercalated divalent cations 508B.

In this example implementation, while under zero or minimal stress, shown in the minimized stress region (#1) in FIG. 5B, the 1° network and the 2° network ion-ion junctions are below the covalent and ionic crosslink burst pressure. Covalent bonds between several 1° networks give the mesh size (0 of the polymer which determines hydrogel swelling and mechanical characteristics. Long crosslinks between 1° networks)(1°-1° produce soft, compliable, elastic gels while short 1°-1° crosslinks bear firm, springy, and rigid gels. Additional strength is achieved by entanglement of the 1° networks that give the HIP extra strength. Emphasis on 1° network free radical quenching is exhibited by indicating free radical termination of 1° network chains. Divalent ion-ion junctions)(2°-2° are also displayed to implicate P(SA) G-block zipping and unzipping while dispersing localized stresses.

FIG. 5C represents the localized stress region #2 (labeled 500C) at crack but before crack propagation. In FIG. 5C, 503C represents tensile force streams applied to the 2° network ions junctions that unzip and dissipate the load from the tensile force streams 503C. The tensile force streams 503C cause the 2° network to travel as shown by 501C with also gives rise to the dissociation of the divalent cations 504C. Together this minimizes the load on the intact crosslinks 505C.

At the localized stress site (#2) in FIG. 5C, the 1° network elongates and redirects most of the stress concentrated at the crack focal point from the 1° network to the 2° network through force streams (arrows). Overloaded, the 2°-2° junctions dissociate and disperse stored energy throughout the medium, preserving the covalent bonded 1° network. 2° grafts are mobile like the 1° network chains and reform 2°-2° junctions providing extra resistance to shear. Thus, by bridging the crack by sacrificing and regenerating 2°-2° junctions, 1° networks can reach maximum polymer chain elongation previously hampered by poor fracture toughness.

Cyclically elongating and relaxing the HIP without time to recover induces mechanical hysteresis is observed to a lesser extent of P(SA) stress-strain hysteresis but for the same mechanical phenomena. Hysteresis is observed because the 2°-2° junctions break before they can reform, resulting in a more malleable HIP with a broader elastic region and lower fracture toughness. Healing can be expedited using low heat to increase G-block and divalent cation motility to reform 2°-2° junctions. Ultimately approximately all the 2°-2° junctions restore and ameliorate the compromised HIP fracture toughness when given a day to heal in humid conditions.

FIG. 5D represents crack propagation and 1° network lysing at region #3 (labeled 500D). In FIG. 5D, the stress induces dissociation of the cations 501D, and breaks the 1° network bonding 502E. T.F.S. flow along 1° network 502G is while under tensile load, and 2°-2° ion junctions 502H return is once the material is at rest. 502F represents the hydrogel fission.

Creating a tough, compliable, malleable HIP with the desired set of acoustic properties requires tuning the concentrations of initiators, and 1° and 2° networks, for the application. Therefore, it is paramount that the effects of each constituent concentration have on the HIP mechanical characteristics are understood and bounded to avoid adverse brittle and rigid HIPs.

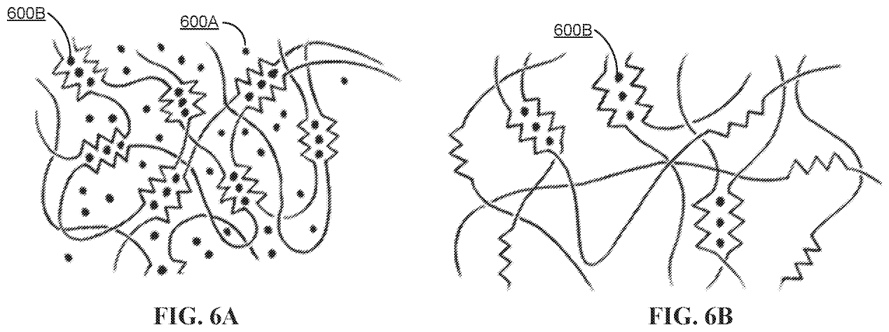

FIGS. 6A and 6B show a diagram depicting a super aggregate hydrogel. For example, sodium alginate is an anionic polysaccharide and block copolymer that crosslinks in the presence of divalent cations via ion-ion junctions and is the HIP sacrificial secondary network grafted to the primary network. Excessive divalent cations create super aggregates; a dense crosslink network inefficient at dispersing energy across the bulk material during plastic deformation, but improves the yield stress of the material at the expense of fracture toughness. In a super aggregate, all G-Blocks in P(SA) are occupied by divalent ions 600B to form ion-ion junctions while extra divalent ions 600A are dissociated in the aqueous, dispersed medium as shown in FIG. 6A. For an infinitesimal divalent ion concentration, few of the G-Blocks are occupied by divalent cations 600B to create ion-ion junctions, forming weak, brittle hydrogels as implicated in FIG. 6B. The few ion-ion junctions are inefficient at dispersing localized stresses, readily “un-zipping” and rapidly propagating cracks through the material.

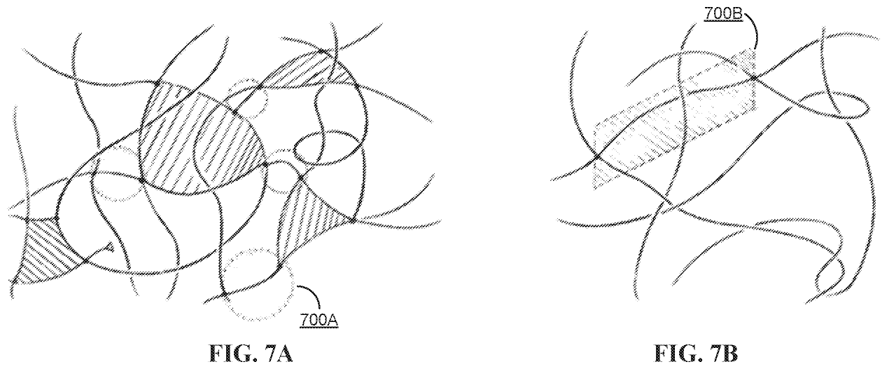

FIGS. 7A and 7B show a diagram depicting a weak, brittle hydrogel. Long poly(DMAm) polymers are composed of reactive allyl-amide monomers polymerized through free-radical vinyl-addition and make up the HIP primary structural network. While propagating, MBA crosslinker react with free radical poly(DMAm) chains, bonding two poly(DMAm) chains together. As more crosslinker is added to solution, the crosslink chain length decreases shown in the circled regions 700A in FIG. 7A. Low molecular weight crosslinks are easier to rupture under stress, resulting in brittle, high modulus poly(DMAm) hydrogels. Too little crosslinker also creates brittle poly(DMAm) hydrogels. Adding minute quantities of crosslinker to solution reduces the number of crosslinks shown in the shaded region 700B in FIG. 7B; as a consequence, there are fewer crosslinks to dissipate forces across the bulk material from localized stress sites, causing the few crosslinks to overload and burst.

Fracture toughness is also optimized by adjusting the concentration of 1° network monomer relative to the 2° network block copolymer. Decreasing the concentration of DMAm to P(SA) increases the elastic modulus while decreasing the fracture toughness and critical stretch. HIPs with more P(SA) exhibits greater resistance to shear from more ion-ion junction interactions, reducing the HIP plasticity and fracture toughness. Increasing the ratio of DMAm to P(SA) decreases the critical stretch, fracture toughness, and elastic modulus, for example. More poly(DMAm) reduces the P(SA) graft density and resistance to material shear; however, with less P(SA) grafts, less sacrificial bonds are available to dissipate localized forces, decreasing the HIP fracture toughness.

Eventually enough sacrificial networks are destroyed and the 1° network maximum elongation is approached which concentrates the force streams along the 1° network. These forces buildup and irreversibly rupture the 1°-1° crosslinks, chain entanglements, and the 1° chain itself with a morphology similar to that presented at the fission line where the hydrogel was cut (#3) as shown in FIG. 5D. After rupturing, 1° and 2° chains are pulled away from one another, denoted by a dashed line, under tensile load and relax after failure. Once relaxed, 2°-2° junctions reform and heal the HIP halves; but, can never restore the HIP along the fission.

In various embodiments, the hydrogels of the present disclosure are structured to create a polymeric matrix having ion junctions for sacrificial networks to dissipate concentrated stress regions and covalent bonds for structural networks that provide elasticity and strength. The 1° network is created via a free radical vinyl addition reaction which governs the elasticity and strength of the HIP.

Free radical vinyl addition chain reactions are initiated when an initiator generates a free radical monomer or free radical chain intermediate that subsequently generates another free radical monomer or chain intermediate. This process continues until most of the free radicals react while the remaining free radicals are unable to react due to physical forces limiting their reaction. The process is summarized below.

a) Initiation:

I→\dot{R}+\dot{R} (1)

M+\dot{R}→\dot{M}₁ (fast) (2)

vᵢ=kᵢ[I] (3)

b) Propagation:

$$\begin{matrix}M + {\dot{M}}_{1} \to {\dot{M}}_{2} & ( 4 ) \\ M + {\dot{M}}_{n - 1} \to {\dot{M}}_{n} & ( 5 ) \\ {v}_{p} = {k}_{p} [ M ] [ \dot{M} ] & ( 6 ) \\ {\overline{v}}_{p} = {( \frac{d [ \dot{M} ]}{d t} )}_{production} = 2 f {k}_{i} [ I ] & ( 7 )\end{matrix}$$

c) Termination:

$$\begin{matrix}{\dot{M}}_{n} + {\dot{M}}_{m} \to {M}_{m + n} ( mutual termination ) & ( 8 ) \\ {\dot{M}}_{n} + {\dot{M}}_{m} \to {M}_{n} + {M}_{m} ( disproportionation ) & ( 9 ) \\ M + {\dot{M}}_{n} \to \dot{M} + {M}_{n} ( chain transfer ) & ( 10 ) \\ {v}_{t} = {{k}_{t} [ \dot{M} ]}^{2} & ( 11 ) \\ {\overline{v}}_{t} = {( \frac{d [ \dot{M} ]}{d t} )}_{depletion} = - 2 {{k}_{t} [ \dot{M} ]}^{2} & ( 12 )\end{matrix}$$

The initiation step is the fast step of the reaction where the initiator (I) dissociates and generates free radicals (\dot{R}) that further generate free radical monomers or chains (M̅). The rate of initiation (vᵢ) is the product of the initiation reaction constant (kᵢ) and the initiator concentration. During the propagation step, free radical chains react with other chains (Mₙ) which in turn become radicalized. The steady state rate of propagation (v̅ₚ) is the product of both terminal chain ends reacting with other chains that, in turn, generate new free radical terminal ends, the rate initiation constant, the concentration of initiator, and the fraction of successful free radical chain initiations (f). The fraction of successful free radical chain initiations is dependent upon the solution temperature, viscosity, and steric inhibition.

Termination can result in one of three ways: mutual termination, disproportionation, and chain transfer. Mutual termination results in longer chain lengths and is thus the desired termination step. Disproportionation results in the termination of free radicals on both chains and results in shorter chain lengths. Chain transfer results in shorter chain lengths for the free radical donor while the free radical receiver becomes chemically active. By assuming chain transfer and disproportionation are minimal, the steady state rate of termination (v̅ₜ) becomes the product of both free radical terminal ends reacting, the termination reaction constant (kₜ), and the concentration of free radical chains.

From the initiation, propagations, and termination steps a net, steady state reaction formula is generated.

d) Net Reaction:

$$\begin{matrix}[ \dot{M} ] = {{( \frac{f {k}_{i}}{{k}_{t}} )}^{0.5} [ I ]}^{0.5} & ( 13 ) \\ {( \frac{d [ \dot{M} ]}{d t} )}_{net} = 2 ( f {k}_{i} [ I ] - {{k}_{t} [ \dot{M} ]}^{2} ) & ( 14 ) \\ {v}_{p} = {{{k}_{p} ( \frac{f {k}_{i}}{{k}_{t}} )}^{0.5} [ I ]}^{0.5} [ M ] = {{k}_{r} [ I ]}^{0.5} [ M ] & ( 15 )\end{matrix}$$

The net rate of propagation (vₚ) is the product of the overall propagation reaction constant (kᵣ), the concentration of the initiator, and the concentration of the chains or monomers present in the solution. Since this is a 1½ order reaction, the monomer concentration (first order) will experience exponential decay in concentration, while the initiator concentration will decay at half the rate of the monomer concentration. Since the free radical chains are less stable than the free radical initiators, the free radical chains react with one another faster than the initiator reacts with the free radical chains. Furthermore, the free radical imitators that react with each other will generate more free radical initiators that will eventually quench when the rate of reaction decreases.

From the rate of propagation, the degree of polymerization ( [symbol] N [symbol] ) and kinetic chain length (v) can be calculated.

e) Degree of Polymerization

$$\begin{matrix}v = \frac{{k}_{p} [ \dot{M} ] [ M ]}{2 {{k}_{i} [ \dot{M} ]}^{2}} = \frac{{k}_{p} [ M ]}{2 {k}_{t} [ \dot{M} ]} = {{k}_{r}^{′} [ \dot{M} ] [ I ]}^{- 0.5} & ( 16 ) \\ {k}_{r}^{′} = ( \frac{1}{2} ) {{k}_{p} ( f {k}_{i} {k}_{t} )}^{- 0.5} & ( 17 ) \\ 〈 N 〉 = 2 v & ( 18 )\end{matrix}$$

The kinetic chain length is the ratio of the rate of chain propagation and the rate production of free radicals; ergo, increasing the concentration of free radicals with respect to the concentration of monomer chains will increase the kinetic chain length. The degree of polymerization for linear chains is directly proportional to the kinetic chain length. As such, an increase in the kinetic chain length yields a two-fold increase in the degree of polymerization.

Because the 1° network is polymerized via vinyl addition reaction, the composition of constituents will have a significant impact on the hydrogel mechanical and acoustic properties. Too much initiator will yield HIPs with extremely short chains that increase the viscosity of the solution but will not create a semi-solid material. On the other hand, too little initiator reduces the rate of reaction to a crawl and can result in higher concentrations of residual monomer if the free radical vinyl addition reaction is quenched before completion.

In a similar manner, excessive catalyst intensifies the rate of initiation and propagation which results in shorter chains lengths resulting in brittle, inelastic HIPs. In turn, minute amounts of catalyst can increase the reaction duration from hours to days. While longer reaction durations can result in longer chain lengths in theory, the increase in solution viscosity during gelation will frequent termination and propagation less and increase the likelihood of oxygen quenching vinyl addition reactions, resulting in HIPs with significant concentrations of residual monomer and free radicals and greater variability in mechanical properties.

Inordinate amounts of monomer for the 1° network give HIPs long chain lengths and strength, but also retain considerable amounts of residual monomer as the reaction proceeds toward gelation which is exacerbated as the solution viscosity increases. On the other extreme, infinitesimal amounts of monomer will lower the rate of propagation and residual monomer concentrations, but generate stiff and brittle HIPs with small kinetic chain lengths because not enough monomer is in solution to create long polymer chains.

Environmental factors like temperature, humidity, and oxygen content should be controlled for consistent mechanical and acoustic properties. Increasing the temperature increases the rate of reaction and reduces the average polymer chain length which makes the HIP brittle and rigid. High relative humidity (RH) can degrade the initiator and catalyst before reacting in solution and reduce the degree of polymerization and increase the amount of residual monomer in the HIP. Moreover, O₂ content in solution can quench free radical vinyl reactions and leave harmful free radicals and residual monomer on the surface of the HIP. Furthermore, the mold vessel has an effect on the surface morphology and chemistry which, in turn, effects the swelling characteristics and biocompatibility of the HIP.

As discussed above, the disclosed hydrogel compositions and articles are engineered to provide certain mechanical and acoustic properties, e.g., including fracture toughness, elasticity, clarity, mechanical tunability, and acoustic tunability, that make the disclosed hydrogels suitable for three-dimensional tomographic ultrasound applications, including ultrasound imaging and Doppler-range diagnostic studies.

Water is an efficient acoustic transmission medium that comprises most of the HIP composition to negligibly attenuate propagated US waves from the transducer. The DIN is the continuous phase composed of the 1° and 2° networks, while water is the dispersed phase in the HIP, resulting in a low bulk modulus semi-solid material that can compress and expand without significant resistance, doubling as an efficient US wave transmission medium.

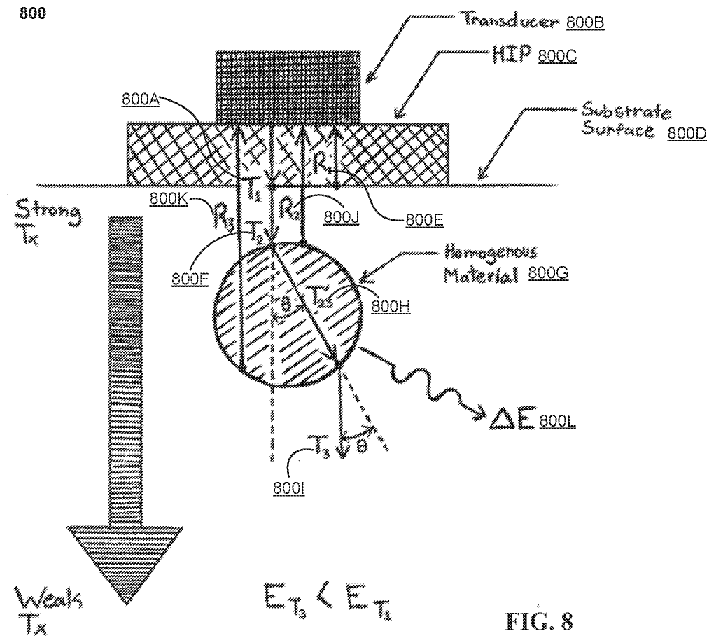

FIG. 8 shows a simple diagram of acoustic transmission through an example HIP into a heterogenous substrate containing homogenous bodies (labeled 800). As shown in the diagram, arrows represent the trajectory of propagated US waves in a medium. Transmitted energy (T) can be refracted (T) and reflected (R) when interacting with material interfaces. Transmitted and refracted energy can be refracted and reflected several times in a medium which scatters the energy. Energy of the transmitted signal (E) is also lost as heat and wave-diffraction, which is summed up with all refraction and scattering potencies through a medium to get the total energy loss of the transmitted signal (ΔE).

At the HIP, substrate/target volume interface, some of the propagated waves are reflected back to the transducer while the rest of the acoustic energy is transmitted into the substrate. Impedance mismatches between the transducer matching layer and the skin interfaces are minimized, only slightly refracting the US waves. Inevitably, the transmitted signal encounters a homogenous body, refracting, diffracting, absorbing, and scattering the transmitted US wave; summed, the total energy loss from the transmitted signal (ΔE) is dispersed throughout the surrounding medium. For each body in the substrate, the transmitted US waves will reflect at the material interfaces and lose energy as aforementioned. There are multiple interfaces, so reducing initial curvature of the US wave propagated through the sonolucent HIP increases the number of reflections and improves the signal-strength of received reflections.

Acoustic transmission is shown in FIG. 8 where the energy denoted by 800A (T₁) is transmitted from the transducer 800B and through the HIP 800C. Some of the energy from 800A (T₁) is reflected back into the transducer 800B at the substrate surface 800D as denoted by 800E (R₁). The rest of the energy denoted by 800F (T₂) is transmitted to the homogenous material 800E. Once the energy 800F (T₂) is transmitted to the homogenous material 800G, the energy 800F (T₂) is transmitted through the homogenous material 800G as well as reflected back into the transducer 800B. For example, some of the energy 800F (T₂), upon entry into the homogenous material 800G, is refracted at an angle (θ) with the resulting refracted energy denoted as 800H (T′₂₃). The refracted energy 800H (T′₂₃) then propagates through the homogenous material 800G and is refracted at an angle (e) as denoted by 8001 (T₃) upon exiting homogenous material 800G. However, some of the energy 800F (T₂), once it propagates through the homogeneous material 800G and contacts the surface of homogenous material 800G, is reflected back into the transducer as denoted by 800K (R₃). Lastly, some of the energy 800F (T₂) does not enter the homogenous material 800D, but rather, is reflected back into the transducer 800B upon contact with the surface of the homogenous material 800G as denoted by 800J (R₂). The total energy lost as denoted by 800L (ΔE) from this process is dispersed throughout the homogenous material 800G.

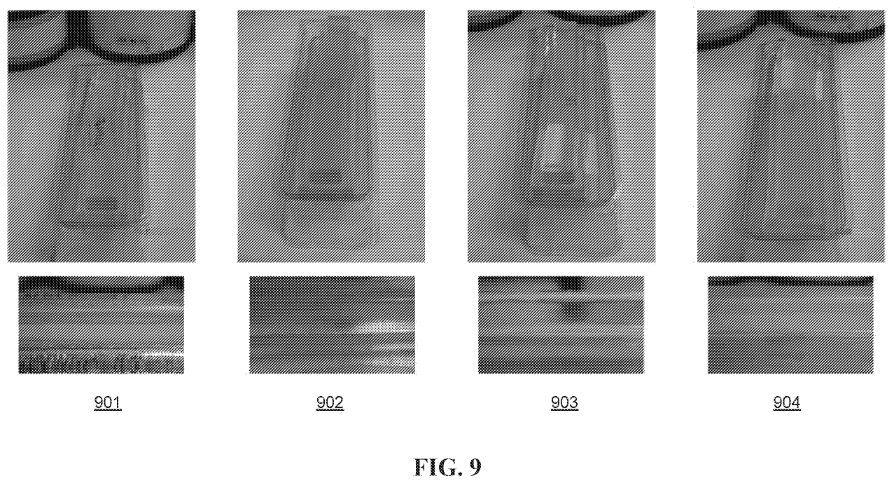

Table 1 show tested acoustic and mechanical properties of example hydrogel compositions for various examples of semi-rigid HIPs in accordance with present technology (hydrogel samples 902, 903, and 904) and for an example control hydrogel sample (901). Note, in Table 1, “SOS” stands for speed of sound; “Z” is acoustic impedance, “ATTN” is attenuation, “E” is the Young's Modulus, and “ε” is the engineering strain. Pictures of the sample hydrogels 901, 902, 903, and 904 are shown in FIG. 9.

TABLE 1

| Hydrogel | SOS | Z | ATTN | | |
|---|---|---|---|---|---|
| Sample (#) | (m/s) | (MRayls) | (dB/cm/MHz) | E (kPa) | ε (mm) |
| 901 | 1548 | 1.595 | 0.14 | 48 | −15 |
| 902 | 1549 | 1.597 | 0.14 | 32 | −15 |
| 903 | 1547 | 1.594 | 0.08 | 46 | −15 |
| 904 | 1547 | 1.594 | 0.03 | 40 | −15 |

Example Implementations of Hydrogel Interface Pads

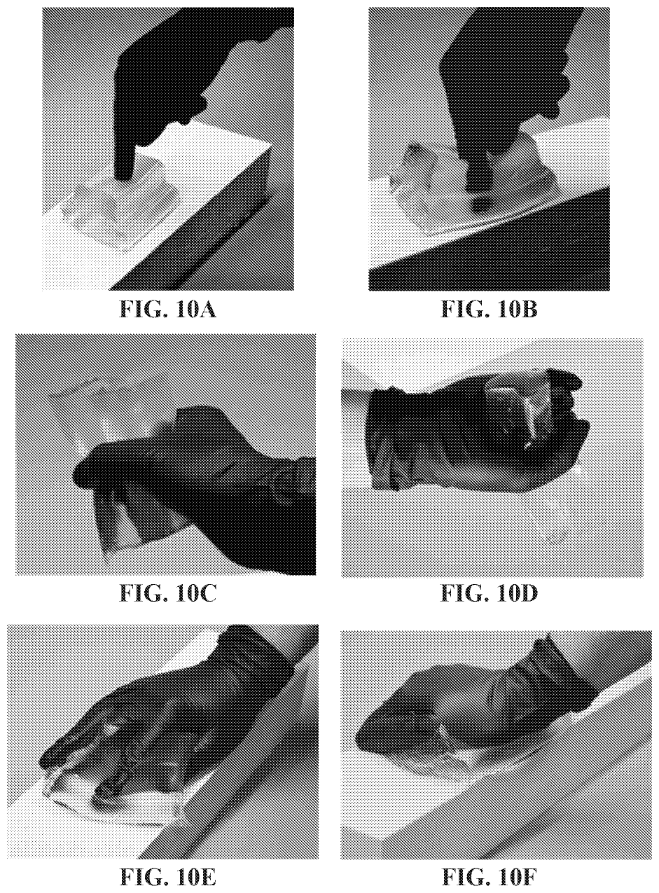

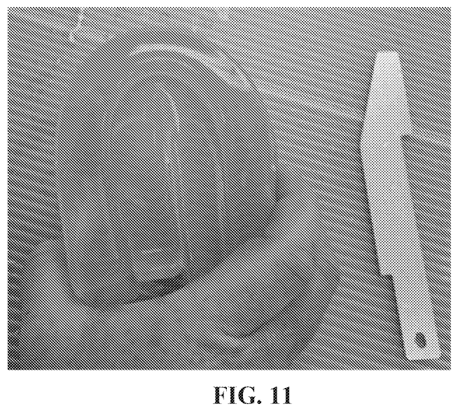

The composition of the HIP has been tailored to create a soft, compliable hydrogel that can conform and envelop the target site to bridge the air acoustic impedance boundary and be tough for clinical applications as demonstrated in FIGS. 10A-10F. By adjusting the HIP composition—covalent and ionic crosslinker, amount and type of 1° network monomer and 2° network block copolymer, and rate of reaction—a range of different mechanical properties can be achieved while maintaining a relatively constant Speed of Sound (SOS), acoustic impedance (Z) and Attenuation (ATTN) as shown in Table 1.

FIGS. 10A-10F show images of the pliability, stretchability, and robustness of the example hydrogel interface pad 903. Specifically, FIG. 10A shows the HIP prior to localized compression, contrasting FIG. 10B which shows the HIP after localized compression. Similarly, FIG. 10C shows the HIP prior to squeezing, contrasting FIG. 10D which shows the HIP after squeezing. Lastly, FIG. 10E shows the HIP conformability characteristics and FIG. 10F shows the HIP under full compression. Taken together, these demonstrations show that the HIP 903 is resistant to fracturing, which is attributed to the toughness and elasticity of the HIP.

In example implementations, the example HIP 901 was used a control hydrogel, composed of Poly(Acrylamide) (Poly(AA)) with low viscosity P(SA) 2° network with good elastic, conformability, and clarity properties. Rippling on HIP 901 exposed surface was due to surface tension differentials during the gelation process. The example HIP 903 was configured to have the same composition as HIP 901 without surface rippling. The example HIP 904 was configured to have the same composition of Poly(AA) and P(SA) components as HIP 901 and HIP 903; but, the example HIP 904 supplements low viscosity P(SA) with high viscosity P(SA). The example HIP 902 was configured to have the same composition of P(SA) as HIP 901 and HIP 903 while substituting Poly(DMAm) for Poly(AA). In these implementations, it was shown that all of the example HIPs had similar acoustic properties while only differentiating in elastic modulus (E) and Ultimate tensile strength (UTS).

For example, rippling on the transducer side of HIP 901 was due to interfacial tension between the air and solution boundary during gelation, causing the gel surface to buckle and warp. HIP 903 reduced the interfacial surface tension during gelation, negating all rippling. HIP 904 supplemented low viscosity P(SA) with high viscosity P(SA) which reduced the elastic modulus considerably, yielding a softer, more pliable HIP. The most pliable was the HIP 902 which had the lowest elastic modulus while exhibiting similar toughness and acoustic energy transmission properties. By further tuning HIP 902 crosslinking rate of reaction, processing variables, and the concentration and types of constituents, a variety of different mechanical properties can be achieved for a plethora of US examination applications without sacrificing good acoustic transmission. As an extreme example, a variant of HIP 902 (HIP 902′) had the same SOS, ATTN, and Z as HIP 902 was overly crosslinked to yield a stiff and bendable hydrogel as shown in FIG. 11. Adding excessive divalent ion crosslinker did not affect the SOS (e.g., 1549 m/s), ATTN (e.g., 0.07 dB/cm*MHz), and clarity while exhibiting an elastic modulus (e.g., 302 kPa) drastically different from HIP 902.

In some implementations, the example hydrogel interface pad can be coupled to an acoustic transducer probe device (e.g., ultrasound scanner). Details of example embodiments of an acoustic transducer probe device that can attach and utilize the example HIP are described in U.S. Publication No. 2016/0242736A1, titled “ACOUSTIC SIGNAL TRANSMISSION COUPLANTS AND COUPLING MEDIUMS,” which is incorporated by reference, in its entirety, as part of the disclosure of this patent document for all purposes.

Example Methods for Fabricating Hydrogel Compositions and Articles

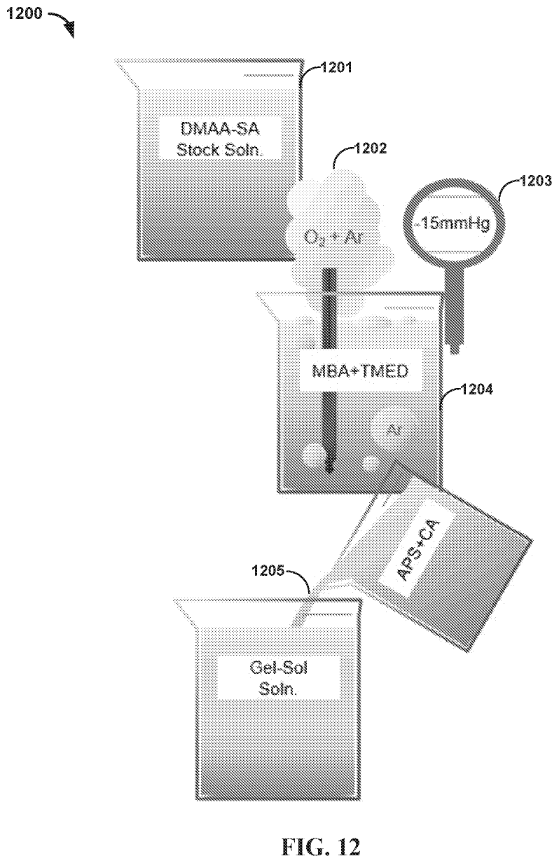

FIG. 12 shows an example embodiment of a method for producing a hydrogel interface pad in accordance with the present technology. The exemplary method, labeled 1200, includes a process 1201 in which DMAm is dissolved in deionized (DI) water at standard temperature and pressure (STP). The addition of DMAm to DI water is an endothermic mixing process in which the DMAm is quickly dissolved. Process 1201 then includes introducing SA to the DMAm solution. Upon the addition of SA to the DMAm solution, the solution swells, resulting in an aggregate/fisheye formation with gelled P(SA) encapsulating dry P(SA) powder. In some implementations the process 1201 of the method, for example, the DMAm-SA serves to form a “stock-solution” for the subsequent reactions and maintains stability over an extended period of time (e.g., greater than 30 min). The method then includes a process 1202 in which the DMAm-SA solution is sparged and degassed with gaseous argon (Ar), nitrogen (N₂), helium (He) or a mixture thereof to remove trace amounts of oxygen (O₂). Next, the method includes a process 2013 in which the solution is placed under reduced pressure in a vacuum chamber to remove all exogenous gases. A solution of degassed N,N′-methylenebisacrylamide (MBA) and N′,N′,N,N-tetramethylethylenediamine (TMED) is then introduced to the DMAm-SA solution to make a “primed-solution” ready for the subsequent polymerization processing steps as shown by process 2014. All solutions need to be void of O₂ otherwise free radical quenching and catalyst oxidation will occur. Lastly, the method includes a process 1205 in which a degassed solution of APS and CA is added to the primed-solution to initiate the exothermic polymerization and crosslinking reactions, thus forming the “gel-solvent”, or “gel-sol”, solution. After the addition of the APS and CA solution, the gel-sol is cast in a mold and cured for less than 8 hours to form the HIP.

Table 2 shows example weight percent ratios (w/w) of each of the components added during the processing step, with their corresponding function for the hydrogel.

TABLE 2

| | Composition | |
|---|---|---|
| Component | (w/w ratios) | Function |
| Sodium Alginate block | 0.5163-5.5297 | Secondary, Grafted |
| copolymer (P(SA)) | | Sacrificial Network |
| Dimethylacrylamide monomer | 3.300-14.830 | Primary, Structural |
| (DMAm) | | Network |
| N,N′-Methylenebisacrylamide | 0.0415-3.4353 | Poly(DMAAm) |
| (MBA) | | Covalent |
| | | Crosslinker |
| N′,N′,N,N- | 0.00426-0.0819 | Catalyst/Promoter |
| Tetramethylethylenediamine | | |
| (TMED) | | |
| Calcium Sulfate (CA), | 0.1382-0.23234 | SA cationic |
| saturated | | crosslinking agent |
| Ammonium Persulfate (APS) | 0.0198-0.235 | Free radical initiator |
| DI water, degassed | 75.655-95.98 | Dispersed Phase |

In some embodiments, the resulting gel-sol exhibits a longer propagation reaction step (i.e., has an increased pot-life). The pot-life has important implications in both the manufacturing of the HIPs on both small and bulk scales as well as slowing down and/or preventing premature polymerization of the constituents from occurring. For example, increasing the pot-life of the gel-sol increases the amount of time for entrained air bubbles to escape the gel-sol after casting the HIP, minimizing the risk of entrapped air in the cured HIP. This characteristic is significant as a failure to remove and/or prevent bubble formation results in poor acoustic transmission—increased attenuation, less reflections, unwanted scattering, and obscured ultrasound images—and compromised mechanical properties—localized stress regions, lower tear strength, and lower burst pressure.

The mesh size of a hydrogel is dependent on various parameters like the reaction rate, chain length, stereochemistry, intramolecular interactions, and reaction conditions such as temperature, pressure, and the atmosphere.

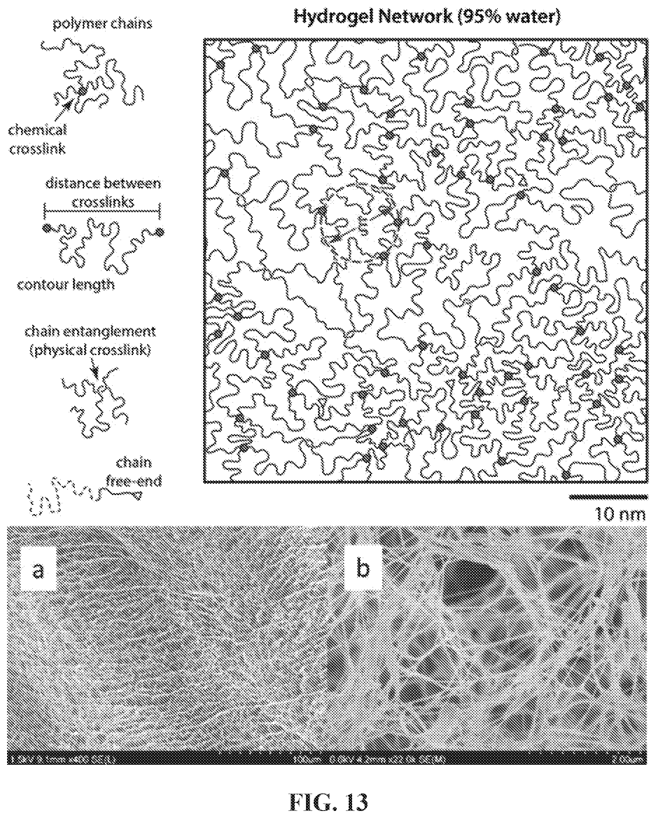

FIG. 13 depicts the mesh of an example Poly(DMA) hydrogel network comprising 95% water and the corresponding electron microscopy images. The mesh of a hydrogel influence clarity, density, SOS, impedance, stiffness, strength, and equilibrium degree of swelling.

In some implementations of the method 1200, for example, an important consideration in making a HIP involves a judicious choice in the quantity of the both the cationic crosslinking agent and the covalent crosslinking agent. For example, in the present exemplary method of making the HIP, the addition of too much of CA (i.e., cationic crosslinking agent) results in the formation of super-aggregates whereas too little CA results in the formation super-dispersions. The equilibrium of too little CA (e.g., super-dispersions) and too much CA (i.e., super-aggregates) is depicted in FIG. 4D. Similarly, too much MBAm (i.e., covalent crosslinking agent) results in a tiny mesh size whereas too little MBAm results in too large of a mesh size. As such, the 0.1382-0.23234 wt % of CA and the 8.290-9.815 wt % of MBAm used in the fabrication of the HIP 903 of the present disclosure provided an optimal degree of aggregation and mesh size.

An additional consideration, crucial to fabricating pliable and robust HIPs, is the degree of grafting that occurs upon reacting the secondary, grafted sacrificial network component (e.g., sodium alginate) and the primary, structural network component (e.g., DMAm). Grafting provides impact strength, energy dissipation, self-healing properties, mechanical hysteresis, and thermal hysteresis of the HIP. The exemplary HIP of the present disclosure exhibits and optimal degree of grafting between the SA and DMAm that affords the aforementioned characteristics.

Yet, for some acoustic signal propagation applications, it may be advantageous to utilize an acoustic couplant with acoustic impedance matching properties that is more rigid and less flexible than at least some of the earlier embodiments described above. For example, such rigid acoustic couplants can include a hydrogel composition that is useful for some ultrasound techniques and applications, e.g., particularly for ultrasound imaging techniques utilizing a flat transducer array and a flat surface of a target volume, where conformance of the couplant to the receiving body is not as great of a challenge.

In some embodiments, a rigid acoustic coupling medium includes a hydrogel composition that comprises dimethylacrylamide monomer (DMAm), where the DMAm is present in an amount of about 1.00 wt % to about 40.00 wt %. In some embodiments of the hydrogel composition, the DMAm is present in an amount of about 3.3 wt % to about 14.8 wt %.

In some embodiments, a rigid acoustic coupling medium includes a hydrogel composition that comprises sodium alginate block copolymer (P(SA)), where the P(SA) is present in an amount of about 0.5 wt % to about 25 wt %. In some embodiments of the hydrogel composition, the P(SA) is present in an amount of about 0.5 wt % to about 5.5 wt %.

In some embodiments, a planar acoustic coupling medium includes a hydrogel composition that comprises dimethylacrylamide monomer (DMAm), where the DMAm is present in an amount of about 1.00 wt % to about 40.00 wt %. In some embodiments of the hydrogel composition, the DMAm is present in an amount of about 3.3 wt % to about 14.8 wt %. In such embodiments, the planar acoustic coupling medium can exhibit brittle and/or weak mechanical properties, e.g., where the material undergoes significant load with or without significant elastic strain before precipitously failing as a small crack suddenly appears and propagates across the material. The significant load can include compressive stress and tensile stress. For example, the compressive stress before failure for the planar hydrogel comprising DMAm can include a range of about 100 kPa to about 250 kPa at 50-80% compression. For example, the tensile stress before failure for the planar hydrogel comprising DMAm can include a range of about 12.0 kPa to about 20.0 kPa at 25-50% elongation. Also, for example, under elastic deformation the planar hydrogel comprising DMAm can suddenly fail.

Yet, despite exhibiting potentially brittle and/or weak mechanical properties, DMAm hydrogels can be durable for some conditions and useful in some applications. For example, if a tensile or shear load is applied to DMAm hydrogels, these hydrogels can suddenly fail between 25-50% strain; yet, under compression, DMAm hydrogels can undergo strains greater than 50% before bursting. In addition, DMAm hydrogels are typically sticky, which can be ideal for situations where the gel must couple to a surface for certain applications, like in long, static ultrasound examinations. Also, for example, for a lumbar spine surgery utilizing laparoscopic ultrasound-guided instruments, a large sheet of DMAm hydrogel could be unfurled to cover the patient's thoracic and lumbar vertebrae while the patient is prone. Because the gel is easily punctured, laparoscopic tools lubricated with sterile water can pierce through the hydrogel and then the tissue, forming a sterile, sonolucent blanket to guide the instruments to the VOL During the surgery, the DMAm hydrogel sticks to the flat topography of the back while the ultrasound-device transmits data regarding the tools proximities to other blood vessels and organs. Notably, a more conformable hydrogel interface pad could also be used with the same amount of success, but would provide significantly more tensile and shear strength.

In some embodiments, a planar acoustic coupling medium includes a hydrogel composition that comprises sodium alginate block copolymer (P(SA)), where the P(SA) is present in an amount of about 0.5 wt % to about 25 wt %. In some embodiments of the hydrogel composition, the P(SA) is present in an amount of about 0.5 wt % to about 5.5 wt %. In some embodiments, the planar acoustic coupling medium can exhibit brittle and/or weak mechanical properties. In such embodiments, the planar acoustic coupling medium exhibits brittleness when it undergoes a significant load, with or without an elastic strain, before failing due to formation and propagation of a crack or crack across the hydrogel material as a result of the significant load. The significant load can include compressive stress and tensile stress. For example, the compressive stress before failure for the hydrogel comprising the alginate can include a range of about 200 kPa to about 500 kPa at 20-60% compression. For example, the tensile stress before failure for the hydrogel comprising alginate can include a range of about 4.5 kPa to about 10.0 kPa at 2-20% elongation. Also, for example, under elastic deformation the hydrogel comprising alginate can suddenly fail.

For example, there are practical applications where a more rigid P(SA) hydrogel would be applicable for scanning planar regions of the body with linear arrays. In particular, because the P(SA) hydrogel is non-tacky, a P(SA) standoff could easily glide over the patient's skin which is ideal for dynamic ultrasound-examinations. For example, a patient with a broken rib could be scanned with a linear array with a rigid P(SA) standoff to scan the ribs near the surface of the skin. If a probe was used without a standoff the rib surface could not be differentiated from the other tissues because of the close proximity of the rib to the transducer. With a standoff, the ultrasound-beam can focus on the bone past the skin surface, generating a crisp picture of the bone underneath the dermal, fat, and ligament layers. Thus, a rib fracture can be easily identified with a medical ultrasound device instead of more expensive and cumbersome CT and MM scans. The more conformable HIP could also be used in this case, but extra conformability provides little to no extra benefit because the array is linear and the patient topography around the ribs is relatively flat.

### Examples

The following examples are illustrative of several embodiments of the present technology. Other exemplary embodiments of the present technology may be presented prior to the following listed examples, or after the following listed examples.

In some embodiments in accordance with the present technology (example 1), a hydrogel composition includes sodium alginate block copolymer (P(SA)), dimethylacrylamide monomer (DMAm), and water, wherein the P(SA) is present in an amount of about 0.5 wt % to about 25.00 wt %, the DMAm is present in an amount of about 1.00 wt % to about 40.00 wt %, and the water is present in an amount of at least about 50.00 wt % of the total weight of the hydrogel composition.

Example 2 includes the hydrogel composition of any of examples 1-10, wherein the DMAm is present in an amount of about 3.3 wt % to about 14.8 wt %.

Example 3 includes the hydrogel composition of any of examples 1-10, wherein the DMAm is present in an amount of about 8.3 wt % to about 9.8 wt %.

Example 4 includes the hydrogel composition of any of examples 1-10, wherein the P(SA) is present in an amount of about 0.5 wt % to about 5.5 wt %.

Example 5 includes the hydrogel composition of any of examples 1-10, wherein the water is present in an amount of at least about 75.6 wt % of the total weight of the hydrogel composition.

Example 6 includes the hydrogel composition of any of examples 1-10, wherein the hydrogel composition further includes N,N′-methylenebisacrylaminde (MBA), N′,N′,N,N-tetramethlethylenediamine (TMED), calcium sulfate (CA), and ammonium persulfate (APS).

Example 7 includes the hydrogel composition of any of examples 1-10, wherein the MBA is present in an amount of about 0.04 wt % to about 10.00 wt % of the total weight of the hydrogel composition.

Example 8 includes the hydrogel composition of any of examples 1-10, wherein TMED is present in an amount of 0.004 wt % to about 1.00 wt % of the total weight of the hydrogel composition.

Example 9 includes the hydrogel composition of any of examples 1-10, wherein CA is present in an amount of about 0.01 wt % to about 1.00 wt % of the total weight of the hydrogel composition.

Example 10 includes the hydrogel composition of any of examples 1-9, wherein the APS is present in an amount of about 0.01 wt % to about 1.00 wt % of the total weight of the hydrogel composition.

In some embodiments in accordance with the present technology (example 11), a semi-rigid acoustic coupling medium includes a hydrogel material, the hydrogel material comprising: a sodium alginate block copolymer (P(SA)), a dimethylacrylamide monomer (DMAm), and water.

Example 12 includes the semi-rigid acoustic coupling medium of any of examples 11-19, wherein the P(SA) is present in an amount of about 0.5 wt % to about 25.00 wt %, the DMAm is present in an amount of about 1.00 wt % to about 40.00 wt %, and the water is present in an amount of at least about 50.00 wt % of the total weight of the hydrogel composition.

Example 13 includes the semi-rigid acoustic coupling medium of any of examples 11-19, wherein the P(SA) is present in an amount of about 0.5 wt % to about 5.5 wt %, the DMAm is present in an amount of about 3.3 wt % to about 14.8 wt %, and the water is present in an amount of at least about 75.6 wt % of the total weight of the hydrogel composition.

Example 14 includes the semi-rigid acoustic coupling medium of any of examples 11-19, wherein the semi-rigid acoustic coupling medium has a speed of sound of 1480-1700 m/s.

Example 15 includes the semi-rigid acoustic coupling medium of any of examples 11-19, wherein the semi-rigid acoustic coupling medium as an acoustic impedance of about 1.00-2.00 MRayls.

Example 16 includes the semi-rigid acoustic coupling medium of any of examples 11-19, wherein the semi-rigid acoustic coupling medium has an acoustic attenuation of about 0.001-1.00 dB/cm/MHz.

Example 17 includes the semi-rigid acoustic coupling medium of any of examples 11-19, wherein the semi-rigid acoustic coupling medium has a Young's Modulus of about 500.00 kPa or less.

Example 18 includes the semi-rigid acoustic coupling medium of any of examples 11-19, wherein the hydrogel has an engineering compressive and elastic strain greater than or equal to 50%.

Example 19 includes the semi-rigid acoustic coupling medium of any of examples 11-18, wherein the hydrogel material further includes N,N′-methylenebisacrylaminde (MBA), N′,N′,N,N-tetramethlethylenediamine (TMED), calcium sulfate (CA), and ammonium persulfate (APS).

In some embodiments in accordance with the present technology (example 20), a hydrogel composition includes sodium alginate block copolymer (P(SA)), dimethylacrylamide monomer (DMAm), and water, wherein the P(SA) is present in an amount of about 0.5 wt % to about 5.5 wt %, the DMAm is present in an amount of about 3.3 wt % to about 14.8 wt %, and the water is present in an amount of at least about 75.6 wt % of the total weight of the hydrogel composition.

Example 21 includes the hydrogel composition of any of examples 20-26, wherein the DMAm is present in an amount of about 8.3 wt % to about 9.8 wt %.

Example 22 includes the hydrogel composition of any of examples 20-26, wherein the hydrogel composition further includes N,N′-methylenebisacrylaminde (MBA), N′,N′,N,N-tetramethlethylenediamine (TMED), calcium sulfate (CA), and ammonium persulfate (APS).

Example 23 includes the hydrogel composition of any of examples 20-26, wherein the MBA is present in an amount of about 0.04 wt % to about 3.4 wt % of the total weight of the hydrogel composition.

Example 24 includes the hydrogel composition of any of examples 20-26, wherein TMED is present in an amount of 0.004 wt % to about 0.082 wt % of the total weight of the hydrogel composition.

Example 25 includes the hydrogel composition of any of examples 20-26, wherein CA is present in an amount of about 0.13 wt % to about 0.23 wt % of the total weight of the hydrogel composition.

Example 26 includes the hydrogel composition of any of examples 20-25, wherein the APS is present in an amount of about 0.02 wt % to about 0.24 wt % of the total weight of the hydrogel composition.

In some embodiments in accordance with the present technology (example 27), a hydrogel composition includes dimethylacrylamide monomer (DMAm), wherein the DMAm is present in an amount of about 1.00 wt % to about 40.00 wt %.

Example 28 includes the hydrogel composition of example 27, wherein the DMAm is present in an amount of about 3.3 wt % to about 14.8 wt %.

In some embodiments in accordance with the present technology (example 29), a hydrogel composition includes sodium alginate block copolymer (P(SA)), wherein the P(SA) is present in an amount of about 0.5 wt % to about 25 wt %.

Example 30 includes the hydrogel of example 29, wherein the P(SA) is present in an amount of about 0.5 wt % to about 5.5 wt %.

In some embodiments in accordance with the present technology (example 31), a planar hydrogel includes dimethylacrylamide monomer (DMAm), wherein the DMAm is present in an amount of about 1.00 wt % to about 40.00 wt %.

Example 32 includes the planar hydrogel of example 31, wherein the DMAm is present in an amount of about 3.3 wt % to about 14.8 wt %.

Example 33 includes the planar hydrogel of any of examples 31 or 32, wherein the planar hydrogel exhibits brittleness when the planar hydrogel undergoes a significant load, with or without an elastic strain, before failing due to formation and propagation of a crack or crack across the planar material as a result of the significant load. The significant load can include compressive stress and tensile stress. For example, the compressive stress before failure for the planar hydrogel comprising DMAm can include a range of about 100 kPa to about 250 kPa at 50-80% compression. For example, the tensile stress before failure for the planar hydrogel comprising DMAm can include a range of about 12.0 kPa to about 20.0 kPa at 25-50% elongation. Also, for example, under elastic deformation the planar hydrogel comprising DMAm can suddenly fail.

In some embodiments in accordance with the present technology (example 34), a planar hydrogel includes sodium alginate block copolymer (P(SA)), wherein the P(SA) is present in an amount of about 0.5 wt % to about 25 wt %.

Example 35 includes the planar hydrogel of example 34, wherein the P(SA) is present in an amount of about 0.5 wt % to about 5.5 wt %.

Example 36 includes the planar hydrogel of any of example 34 or 35, wherein the planar hydrogel exhibits brittleness when the planar hydrogel undergoes a significant load, with or without an elastic strain, before failing due to formation and propagation of a crack or crack across the planar material as a result of the significant load. The significant load can include compressive stress and tensile stress. For example, the compressive stress before failure for the planar hydrogel comprising the alginate can include a range of about 200 kPa to about 500 kPa at 20-60% compression. For example, the tensile stress before failure for the planar hydrogel comprising alginate can include a range of about 4.5 kPa to about 10.0 kPa at 2-20% elongation. Also, for example, under elastic deformation the planar hydrogel comprising alginate can suddenly fail.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A hydrogel composition, comprising:
sodium alginate block copolymer (P(SA)), dimethylacrylamide monomer (DMAm), and water, wherein
the P(SA) is present in an amount of about 0.5 wt % to about 25.00 wt %,
the DMAm is present in an amount of about 1.00 wt % to about 40.00 wt %, and
the water is present in an amount of at least about 50.00 wt % of the total weight of the hydrogel composition.

2. The hydrogel composition of claim 1, wherein the DMAm is present in an amount of about 3.3 wt % to about 14.8 wt %.

3. The hydrogel composition of claim 1, wherein the DMAm is present in an amount of about 8.3 wt % to about 9.8 wt %.

4. The hydrogel composition of claim 1, wherein the P(SA) is present in an amount of about 0.5 wt % to about 5.5 wt %.

5. The hydrogel composition of claim 1, wherein the water is present in an amount of at least about 75.6 wt % of the total weight of the hydrogel composition.

6. The hydrogel composition of claim 1, further comprising:
N,N′-methylenebisacrylaminde (MBA), N′,N′,N,N-tetramethlethylenediamine (TMED), calcium sulfate (CA), and ammonium persulfate (APS).

7. The hydrogel composition of claim 6, wherein the MBA is present in an amount of about 0.04 wt % to about 10.00 wt % of the total weight of the hydrogel composition.

8. The hydrogel composition of claim 6, wherein TMED is present in an amount of 0.004 wt % to about 1.00 wt % of the total weight of the hydrogel composition.

9. The hydrogel composition of claim 6, wherein CA is present in an amount of about 0.01 wt % to about 1.00 wt % of the total weight of the hydrogel composition.

10. The hydrogel composition of claim 6, wherein the APS is present in an amount of about 0.01 wt % to about 1.00 wt % of the total weight of the hydrogel composition.

11. A semi-rigid acoustic coupling medium comprising a hydrogel material, the hydrogel material comprising: a sodium alginate block copolymer (P(SA)), a dimethylacrylamide monomer (DMAm), and water.

12. The semi-rigid acoustic coupling medium of claim 11, wherein
the P(SA) is present in an amount of about 0.5 wt % to about 25.00 wt %,
the DMAm is present in an amount of about 1.00 wt % to about 40.00 wt %, and
the water is present in an amount of at least about 50.00 wt % of the total weight of the hydrogel composition.

13. The semi-rigid acoustic coupling medium of claim 11, wherein
the P(SA) is present in an amount of about 0.5 wt % to about 5.5 wt %,
the DMAm is present in an amount of about 3.3 wt % to about 14.8 wt %, and
the water is present in an amount of at least about 75.6 wt % of the total weight of the hydrogel composition.

14. The semi-rigid acoustic coupling medium of claim 11, wherein the semi-rigid acoustic coupling medium has a speed of sound of 1480-1700 m/s.

15. The semi-rigid acoustic coupling medium of claim 11, wherein the semi-rigid acoustic coupling medium as an acoustic impedance of about 1.00-2.00 MRayls.

16. The semi-rigid acoustic coupling medium of claim 11, wherein the semi-rigid acoustic coupling medium has an acoustic attenuation of about 0.001-1.00 dB/cm/MHz.

17. The semi-rigid acoustic coupling medium of claim 11, wherein the semi-rigid acoustic coupling medium has a Young's Modulus of about 500.00 kPa or less.

18. The semi-rigid acoustic coupling medium of claim 11, wherein the hydrogel has an engineering compressive and elastic strain greater than or equal to 50%.

19. The semi-rigid acoustic coupling medium of claim 11, wherein the hydrogel material further comprises:
N,N′-methylenebisacrylaminde (MBA), N′,N′,N,N-tetramethlethylenediamine (TMED), calcium sulfate (CA), and ammonium persulfate (APS).

20. A hydrogel composition, comprising:
sodium alginate block copolymer (P(SA)), dimethylacrylamide monomer (DMAm), and water, wherein
the P(SA) is present in an amount of about 0.5 wt % to about 5.5 wt %,
the DMAm is present in an amount of about 3.3 wt % to about 14.8 wt %, and
the water is present in an amount of at least about 75.6 wt % of the total weight of the hydrogel composition.

21.-36. (canceled)
